(19) United States
(12) Patent Application Publication
(10) Pub. No.: US 2013/0058724 A1
(43) Pub. Date: Mar. 7, 2013

(54) NOVEL MULTIFUNCTIONAL MATERIALS FOR IN-SITU ENVIRONMENTAL REMEDIATION OF CHLORINATED HYDROCARBONS

(71) Applicants: Vijay John, Destrehan, LA (US); Noshir Pesika, River Ridge, LA (US); Gerhard Piringer, Vienna (AT); Jingjing Zhan, New Orleans, LA (US); Gary McPherson, Mandeville, LA (US)

(72) Inventors: Vijay John, Destrehan, LA (US); Noshir Pesika, River Ridge, LA (US); Gerhard Piringer, Vienna (AT); Jingjing Zhan, New Orleans, LA (US); Gary McPherson, Mandeville, LA (US)

(73) Assignee: THE ADMINISTRATORS OF THE TULANE EDUCATIONAL FUND, New Orleans, LA (US)

(21) Appl. No.: 13/502,047

(22) PCT Filed: Oct. 14, 2010
(86) PCT No.: PCT/US10/52713

§ 371 Date: Nov. 19, 2012

Related U.S. Application Data
(60) Provisional application No. 61/251,632, filed on Oct. 14, 2009.

(51) Int. Cl. B09C 1/08; C07C 1/26; C09K 3/32; B82Y 30/00

(52) U.S. Cl. 405/128.5; 252/184; 585/254; 977/773; 977/903

(57) ABSTRACT

Effective in-situ injection technology for the remediation of dense nonaqueous phase liquids (DNAPLs) such as trichloroethylene (TCE) benefits from the use of decontamination agents that effectively migrate through the soil media, and react efficiently with both dissolved TCE and bulk TCE. A novel decontamination system contains highly uniform carbon microspheres preferably in the optimal size range for transport through the soil. The microspheres are preferably enveloped in a polyelectrolyte (such as carboxymethyl cellulose, CMC) to which preferably a bimetallic nanoparticle system of zerovalent iron and Pd is attached. The carbon serves as a strong adsorbent to TCE, while the bimetallic nanoparticles system provides the reactivity. The polyelectrolyte serves to stabilize the carbon microspheres in aqueous solution. The overall system resembles a colloidal micelle with a hydrophilic shell (the polyelectrolyte coating) and a hard hydrophobic core (carbon). In contact with bulk TCE, there is a sharp partitioning of the system to the TCE side of the interface due to the hydrophobicity of the core. These multifunctional systems appear to satisfy criteria related to remediation and are relatively inexpensive and made with potentially environmentally benign materials. An aerosol process is preferably used to produce zerovalent iron particles supported on carbon. A method of lubricating includes creating carbon microspheres produced from a monosaccharide or polysaccharide, the carbon microspheres having a diameter of 50 nm to 6 microns, coating the microspheres with a surface coating and using the carbon microspheres as a lubricant.

### CROSS-REFERENCE TO RELATED APPLICATIONS

Incorporated herein by reference is our U.S. Provisional Patent Application No. 61/251,632, filed 14 Oct. 2009, priority of which is hereby claimed.

### STATEMENT REGARDING FEDERALLY SPONSORED RESEARCH OR DEVELOPMENT

The present invention was funded in part by a grant from the Environmental Protection Agency (EPA-GR832374) and in part by a grant from National Science Foundation grant No. 0933734. The United States government has certain rights in this invention.

### REFERENCE TO A “MICROFICHE APPENDIX”

Not applicable

### BACKGROUND OF THE INVENTION

1. Field of the Invention

The primary uses and commercial applications of this invention are in environmental remediation technologies. There is a huge market for new environmental remediation methods that dispose of chlorinated hydrocarbons.

Conventional technology either attempts to use zerovalent iron nanoparticles or coats these particles with polymers. The coating methods are cost prohibitive and the polymers may not be environmentally benign. The use of biodegradable polymers and proteins as coatings has been proposed but here again, it is not known whether these coatings will survive the transport through sediments.

Effective in-situ injection technology for the remediation of dense nonaqueous phase liquids (DNAPLs) such as trichloroethylene (TCE) requires the use of decontamination agents that effectively migrate through the soil media, and react efficiently with both dissolved TCE and bulk TCE.

The present invention includes the use of a novel decontamination system containing highly uniform carbon microspheres in the optimal size range for transport through the soil. The microspheres are preferably enveloped in a polyelectrolyte (carboxymethyl cellulose, CMC) to which a bimetallic nanoparticle system of zerovalent iron and Palladium (Pd) is preferably attached. We can also use Platinum (Pt), Gold (Au), and Nickel (Ni) instead of Pd that have been mentioned in the literature. Ni is the least expensive. Pd is the best to use, however, Ni is close to Pd. We can also use a range of transition metals, of which Ni is one. There is evidence in the prior art that states that Ni can be used. The carbon serves as a strong adsorbent to TCE, while the bimetallic nanoparticles system provides the reactivity. The polyelectrolyte serves to stabilize the carbon microspheres in aqueous solution. The overall system resembles a colloidal micelle with a hydrophilic shell (the polyelectrolyte coating) and a hard hydrophobic core (carbon). In contact with bulk TCE, there is a sharp partitioning of the system to the TCE side of the interface due to the hydrophobicity of the core. These multifunctional systems appear to satisfy criteria related to remediation and are relatively inexpensive and made with potentially environmentally benign materials.

We have experimental data to show that the particles are effective in breaking down TCE. We also have data that indicate that these materials effectively transport through model sediments.

2. General Background of the Invention

Nanoscale zero-valent iron (ZVI) particles are a preferred option for the reductive dehalogenation of trichloroethylene (TCE). However, it is difficult to transport these particles to the source of contamination due to aggregation. The present invention includes a novel approach to the preparation of ZVI nanoparticles that are efficiently and effectively transported to contaminant sites. The technology developed involves the encapsulation of ZVI nanoparticles in porous sub-micron silica spheres which are easily functionalized with alkyl groups. These composite particles preferably have the following characteristics (1) They are in the optimal size range for transport through sediments (2) dissolved TCE adsorbs to the organic groups thereby bringing tremendously increasing contaminant concentration near the ZVI sites (3) they are reactive as access to the ZVI particles is possible (4) when they reach bulk TCE sites, the alkyl groups extend out to stabilize the particles in the TCE bulk phase or at the water-TCE interface (5) the materials are environmentally benign. We have extensively demonstrated these concepts through reactivity studies, and transport studies using column transport, capillary and microcapillary transport studies. These iron/silica aerosol particles with controlled surface properties also have the potential to be efficiently applied for in situ remediation and permeable reactive barriers construction.

In extensions of the work, it has been shown that these particles function effectively as reactive adsorbents for TCE. The present invention includes the synthesis of such composite nanoscale materials through an aerosol-based method and through solution methods, to illustrate the versatility and ease of materials synthesis, scale up and application. The present invention also includes the development of carbon submicron particles that serve as supports for zerovalent iron with optimal transport and reactivity characteristics.

Chlorinated hydrocarbons such as trichloroethylene (TCE) form a class of dense non-aqueous phase liquid (DNAPL) contaminants in groundwater and soil that are difficult to remediate. They have a density higher than water and settle deep into the sediment from which they gradually leach out into aquifers, causing long term environmental pollution. Remediation of these contaminants is of utmost importance for the cleanup of contaminated sites [1-3]. In recent years, the reductive dehalogenation of such compounds using zerovalent iron (ZVI) represents a promising approach for remediation. The overall redox reaction using TCE as an example is

C₂HCl₃+4Fe⁰+5H⁺→C₂H₆+4Fe²⁺+3Cl⁻

where gaseous products such as ethane result from complete reduction. The environmentally benign nature of ZVI and its low cost are attractive to the development of such remediation technologies. Compared to more conventional treatment processes, the in-situ direct injection of reactive zero-valent iron into the contaminated subsurface is a preferred method because it may more directly access and target the contaminants [4, 5]. Nanoscale zero-valent iron particles (NZVI) often have higher remediation rates resulting from their increased surface area [6-14]. More importantly, the colloidal nature of nanoiron indicates that these particles can be directly injected into contaminated sites for source depletion or, alternatively, be devised to construct permeable reactive barriers for efficient TCE remediation [15-21].

For successful in-situ source depletion of pure phase TCE, it is believed to be best for injected nanoscale ZVI to migrate through the saturated zone to reach the contaminant; one can consider successful strategies the environmental equivalent of targeted drug delivery. The transport of colloidal particles such as nanoiron through porous media is determined by competitive mechanisms of diffusive transport, interception by soil or sediment grains and sedimentation effects as shown through the now-classical theories of colloid transport [18, 22-25]. The Tufenkji-Elimelch model [26] which considers the effect of hydrodynamic forces and van der Waals interactions between the colloidal particles and soil/sediment grains is a significant advance in modeling transport of colloidal particles through sediment, and predicts optimal particle sizes between 200 nm-1000 nm for zerovalent iron particles at typical groundwater flow conditions [27, 28]. At particle sizes exceeding 15 nm, however, ZVI exhibits ferromagnetism, leading to particle aggregation and a loss in mobility [29]. The particles by themselves are therefore inherently ineffective for in-situ source depletion. One of the common methods to increase nanoiron mobility is to stabilize the particles by adsorption of organic molecules on the particle surface [30-34]. The adsorbed molecules enhance steric or electrostatic repulsions between particles to prevent their aggregation. Techniques include the use of polymers, surfactants, starch, modified cellulose, and vegetable oils as stabilizing layers to form more stable dispersions [27, 32-41]. These methods enhance steric or electrostatic repulsions of particles to prevent their aggregation and may be effective if the physically adsorbed stabilizers are retained during particle migration through sediments. Functionalization of ZVI nanoparticles with organic ligands is another alternative but such functionalization is not easy and it is unclear if the reactivity of ZVI is retained.

FIG. 1 summarizes the objectives behind our recent work where we seek to develop multifunctional nanoscale materials for adsorption, reaction, transport and partitioning. On the left, we show the concept of entrapment of NZVI in porous submicron particles of functionalized silica. The functional groups are typically hydrophobic alkyl groups which, in aqueous solution, stay confined to the silica. The silica particles are designed to have the optimal size range for transport through sediment. As the particles travel through water-saturated sediment following groundwater flow streamlines, there is a significant adsorption of dissolved TCE onto the alkyl groups, thereby bringing the contaminant in close proximity to the NZVI (center). When the composite particles reach a site of bulk TCE, the alkyl groups extend out increasing the hydrodynamic radius of the particle thereby reducing its effective density (right). It is an objective to stabilize these particles either in the TCE bulk or at the water-TCE bulk interface.

The actualizations of these concepts are next presented. In order to separate the NZVI particles, we preferably entrap them in a porous silica matrix, where the silica particles are typically submicron sized. The process of encapsulation is preferably through an aerosol-based process [42-44]. The postulated advantages behind the work are the following: [a] entrapment of ZVI into porous silica may make the ZVI less prone to aggregation, while maintaining reactivity; [b] silica is environmentally benign and entrapment of ZVI into porous silica reduces the safety concerns of nanoiron hazards of fire and explosion when exposed to air [45]; [c] the aerosol-based process is a route to synthesis of porous colloidal silica in the optimal size range for transport; being just a variation of the spray-drying process, scale-up to produce large quantities of the material is feasible; [d] functionalization of silica is extremely simple and there are several methods of silica functionalization that could be exploited to allow maximum contact of ZVI with the contaminant (TCE) [a] alkyl groups are microbially degradable. Point [d] is especially relevant from two perspectives. First, it would be a significant advantage to target the delivery of ZVI so that the particles transport efficiently through the saturated zone and then effectively partition to the water-TCE interface upon encountering regions of bulk TCE. Second, if silica can be functionalized appropriately, the sparingly soluble pure phase TCE in water would partition to the silica, increasing local concentrations and accessibility to the ZVI nanoparticles.

FIG. 2 illustrates the concepts of NZVI encapsulation in porous silica through the aerosol process. In this process, silica precursors such as tetraethyl orthosilicate (TEOS) and ethyl triethoxysilane (ETES) together with iron precursors are aerosolized with the aerosol droplets passing through a high temperature zone. During this process, silicates hydrolyze and condense in the droplet entrapping the iron species. The “chemistry in a droplet” process leads to submicron sized particles of silica containing iron nanoparticles which are then collected on a filter. Since the particles are essentially made with silica and iron they are environmentally benign. Of particular relevance also is the use of alkyl groups attached to the silica through the use of alkyl-silane precursors such as ETES. These groups introduce porosity into the silica. Additionally, these organic groups play an important role in that they serve as adsorbents for the TCE, thus bringing the organic contaminant to the vicinity of the iron species and facilitating reaction. We note that mixtures of ethyltrioxysilane (ETES) and tetraethylorthosilicate (TEOS) lead to particles where the degree of incorporation of the alkyl functionality can be adjusted.

FIG. 3 shows the size distribution of the composite Fe/Ethyl-Silica particles, showing polydispersity that is inherent in the aerosol-based process. The inset shows a TEM indicating zerovalent iron nanoparticles decorating the silica matrix. FIG. 4 illustrates the reactivity characteristics of the composite particles when contacted with dissolved TCE. There is a significant drop in solution TCE concentration followed by a graduate decrease. The initial concentration drop is not due to reaction but to adsorption. This is clearly shown by the gaseous product evolution (ethane and ethylene) which is much more gradual. Additionally, when the composite particles are prepared without the alkyl functional groups, using just TEOS as the silica precursor, the sudden drop in TCE solution concentration is not observed [46].

The adsorptive-reactive concept is extremely important in the design of multifunctional particles. Adsorption leads to high local concentrations in the vicinity of the reactive zerovalent iron, potentially facilitating reaction. We also note that the reaction rate can be enhanced significantly upon deposition of small quantities of Pd through the incorporation of Pd(OAc)₂ in the precursor solution [47]. The catalytic effect of Pd in dramatically enhancing reaction rates has been discussed in detail in the literature [7, 48, 49]. The role of Pd is to dissociatively chemisorb hydrogen produced by redox reactions on Fe⁰. We can also use Platinum (Pt), Gold (Au), Nickel (Ni) instead of Pd that have been mentioned in the literature. Ni is the least expensive.

The particle size range is also important from a transport perspective. Filtration theory predicts that the migration of colloidal particles through porous media such as soil is typically dictated by Brownian diffusion, interception and gravitational sedimentation [50]. The Tufenkji-Elimelech (T-E) model is perhaps the most comprehensive model to describe these effects in the presence of interparticle interactions [26], with the governing equation

η₀=2.4A_{S}^{1/3}N_{R}^{-0.081}N_{Pe}^{-0.715}N_{vdW}^{0.052}+0.55A_{S}N_{R}^{1.675}N_{A}^{0.125}+0.22N_{R}^{-0.24}N_{G}^{1.11}N_{vdW}^{0.053}

where η₀ is the collector efficiency, simply defined as the probability of collision between migrating particles and sediment grains. The first term on the right characterizes the effects of particle diffusion on the collector efficiency, while the second and third terms describe the effects of interception and sedimentation. However, the Tufenkji-Elimelech equation does not provide the complete representation of particle transport, which also involves concepts such as bridging and attachment between the particles and the surfaces of soil grains, characterized through a “sticking coefficient” [39]. For brevity, we limit the discussion of the T-E equation to demonstrating the dependence of the collector efficiency on particle size as shown in FIG. 5. As seen in the figure, the collector efficiency is minimized at a particle size range 0.1 to 1 μm, which implies that this is the optimal size range for colloid particles to migrate through the soil, and is in the size range obtained through the aerosol-based process (FIG. 3). FIG. 5 also indicates an optical micrograph of commercially available ZVI nanoparticles, the reactive nanoiron particles (RNIP-10DS, which is uncoated or bare RNIP) from Toda Kogyo Corporation. While the intrinsic particle size of these particles is of the order 30-70 nm, aggregation to effective sizes over 10 μm make them ineffective for transport through soil [29].

We have carried out column and capillary transport experiments (FIG. 6) on the Fe/Ethyl-Silica particles to determine the transport characteristics [51]. As predicted by the TE equation, the Fe/Ethyl-silica particles elute efficiently through model sediment-packed columns, while the control bare RNIP remains aggregated at the head of the column.

Capillary studies confirm these findings [51]. FIG. 7 illustrates these studies where 1.5 mm horizontal capillaries are filled with sediment and particle transport is visualized through optical microscopy. Here it is clearly evident that the bare RNIP (panel (i) in the middle and panel (i) at the bottom) is retained at the capillary inlet, while visualization of Fe/Ethyl-Silica particles is clearly observed throughout the capillary during transport (panels ii in the middle and the bottom), and at the end of the capillary after elution is complete (iii in the middle). At the beginning of the experiment all system appear the same as (i) in the middle set of panels with a particle suspension at the inlet to the capillary. FIG. 8 illustrates a microcapillary visualization experiment where a TCE droplet is injected using a micropipetter into a 200 μm capillary containing dispersed Fe/Ethyl-Silica particles in water. We see a stable aggregation of the particles on the TCE droplet interface.

These past results summarize our experiments with NZVI supported on novel Fe/Ethyl-Silica particles prepared through the aerosol-based route. The disadvantage of making these materials a reality in environmental remediation is perhaps the cost of the silica precursor (ethyltriethoxysilane and tetraethyl orthosilicate) which becomes an overriding factor in developing applications to environmental remediation. In the present invention we are adapting carbon-based materials to support ZVI nanoparticles.

In 2001, there was a very interesting report published by Wang et al. in Carbon [77] illustrating a novel and simple method to synthesize spherical microporous carbons. These authors took sucrose in solution and subjected it to hydrothermal treatment at 190° C. At these conditions (12 bar vapor pressure), the sugar undergoes dehydration and the resultant material has the morphology of extremely monodisperse carbon spheres. Upon pyrolysis of these materials, graphitic carbon spheres are obtained with sizes ranging from 50 nm to 6 μm (preferably 200 nm to 6 μm; more preferably 200 nm to 1.5 μm; even more preferably 300-700 nm; most preferably 400-600 nm; e.g. 500 nm) depending on the sugar concentrations used. These authors have been studying the materials for applications in Li-ion batteries as they make promising anode materials [52]. We have been able to reproduce the morphologies of these materials as shown in the SEMs in FIG. 9. The particles on the left were prepared using a precursor sucrose solution of 0.15M concentration, while the particles on the right were prepared using a concentration of 1.5M.

These results become easily connected to the environmental problem of TCE remediation. Carbons are environmentally innocuous. The hydrothermal+pyrolysis process is simple and can be easily scaled up as a solution process. The carbon precursors are also inexpensive. Most importantly, the particle size can be tuned for optimal transport characteristics very easily by modifying precursor concentrations.

We have described our research as directed towards the development of zerovalent iron based multifunctional particles with the following characteristics (a) they are reactive to the reduction of chlorinated hydrocarbons (b) they transport through sediments and migrate to the sites of TCE contamination (c) they adsorb TCE, lowering bulk dissolved TCE concentrations and bringing TCE to the proximity of the zerovalent reduction sites (d) they partition to the interface of bulk TCE. Fe/silica composite particles prepared through the aerosol-based route appear to have these characteristics. The aerosol route is not difficult to be scaled up since it is just a variation of traditional spray drying processes. However, the precursor costs may make these materials unattractive for practical application. In the present invention, we are developing carbon based materials that exhibit these characteristics. The novelty of the carbon based approach is the methodology to make uniform sized particles in a simple manner.

Dense non-aqueous phase liquids (DNAPLs) such as trichloroethylene (TCE) have a specific gravity greater than water and migrate deep into the subsurface from which they gradually dissolve into aquifers causing problems of long term environmental pollution. The cleanup of groundwater contaminated with these DNAPLs is a challenging task due to the natural properties of subsurface heterogeneity and complex architecture [1A, 2A]. Extensive efforts have been made to develop methods for the remediation of DNAPLs, and various strategies have been explored such as air sparging/soil vapor extraction, pump-and-treat, installation of permeable reactive barriers and bioremediation [3A-8A]. Compared to these approaches, the in situ injection of nanoscale zerovalent iron (NZVI) to reduce DNAPLs has become a potentially simple, cost-effective, and environmentally benign technology and has become a preferred method in the remediation of these compounds [9A, 10A], where the following redox reaction (shown for TCE)

C₂HCl₃+4 Fe⁰+5H⁺→C₂H₆+4 Fe²⁺+3 Cl⁻

leads to conversion of the contaminant to innocuous gas phase species such as ethane.

For effective in-situ remediation of TCE using NZVI, it is important for the remediation agents/particles to effectively migrate through the soil [11A, 12A]. Bare NZVI particles have a strong tendency to agglomerate due to their high surface energies and intrinsic magnetic interactions, forming aggregates that plug and inhibit their flow through porous media. Prior studies have shown that the mobility of NZVI particles can be enhanced dramatically by adsorption of hydrophilic or amphiphilic organic species such as surfactants, vegetable oils, starch, or polyelectrolytes such as carboxymethyl cellulose (CMC) and poly (acrylic acid) (PAA), or triblock copolymers on the NZVI particle surface [13A-19A]. These adsorbed organics inhibit NZVI aggregation and enhance solution stability through steric hindrance and/or electrostatic repulsion. Alternatively, NZVI has been immobilized onto 1-3 mm activated carbon granules to inhibit aggregation [20A, 21A]. Composites with carbon introduce a strong adsorptive aspect into remediation technology as the carbon adsorbs chlorinated compounds, and these materials have been used in the development of adsorptive-reactive barriers [22A]. Previous research in our laboratory has focused on the development of silica particles containing NZVI nanoparticles prepared through an aerosol-based route [12A, 23A]. These composite particles are in the size range 100 nm-1 μm which is believed to be the optimal size range for effective transport through sediments. The functionalization of these particles with alkyl moieties leads to strong adsorption capacities, and the particles function as adsorbents with coupled reactivity characteristics. However, the possible disadvantage of this process is the cost of silica precursors (ethyltriethoxysilane and tetraethylorthosilicate) used in the aerosol-based process.

Several criteria need to be met in the effective design of multifunctional colloid particulate systems for in-situ TCE degradation. Such particulate systems must be able to move through the subsurface with optimal mobility, reach sites of TCE contamination, partition to the TCE phase and break down the contaminant. While following groundwater flow through the subsurface, it would be advantageous if these particles also reduce the concentration of dissolved TCE through a combination of sequestration by adsorption followed by reaction. Additional factors include the following (1) the use of small amounts of a catalyst, typically Pd, to dramatically enhance reactivity through dissociative adsorption of H₂ on the catalyst surface [11A, 24A-26A] (2) the mobility of colloids in the subsurface is determined by competitive mechanisms of Brownian motion, interception by soil and sediment grains and sedimentation effects. The Tufenkji-Elimelch model, which considers the effect of hydrodynamic forces and van der Waals interactions between colloidal particles and sediment grains predicts that particles in the size range 0.1 to 1.0 microns are likely to be the most mobile at typical groundwater flow conditions [17A, 23A, 27A].

The present invention comprises a new method for designing in-situ remediation by combining two remarkable concepts. The first concept as pioneered by Zhao and coworkers [15A, 18A, 28A], is the use of inexpensive and environmentally benign polymers such as carboxymethyl celluose (CMC-FIG. 22a) which have been found to be effective at nucleating nanoparticles of ZVI and preventing their aggregation [48A]. These polymer stabilized NZVI systems are effective in TCE dechlorination, but the water solubility of the polymer inhibits partitioning to TCE bulk phases, and the polymer exhibits negligible adsorption capacities for TCE. Nevertheless, the ability of these inexpensive systems to prevent NZVI aggregation and to stay suspended in water indicates significant potential in groundwater remediation.

The second concept applied here is the novel technology behind the development of highly uniform monodisperse carbon microspheres through hydrothermal dehydration of simple sugars followed by carbonization. This technology, pioneered by Wang and coworkers has been promoted in the development of carbon electrodes and in electrochemical applications [29A-31A]. We have recognized that these carbon microspheres may be developed into adsorbents much like activated carbons. In addition, the fact that the microspheres are in the optimal size range for transport as predicted by the T-E model and that they can be made with high monodispersity and with inexpensive precursors, provides the motivation to test their use in the in-situ remediation of TCE. FIGS. 11a and 11b are scanning and transmission electron micrographs of such carbons made in our laboratory from sucrose, and the monodispersity of the particles is immediately apparent. Simple variation of precursor concentration results in monodisperse particles with sizes ranging from less than 500 nm to 5 μm (around 50 nm to 6 μm; preferably 200 nm to 6 μm; more preferably 200 nm to 1.5 μm; even more preferably 300-700 nm; most preferably 400-600 nm; e.g. 500 nm) as the precursor concentration is increased tenfold from approximately 0.15M to 1.5 M.

The following are incorporated herein by reference:

Zhao, Dongye; He, Feng. Preparation and applications of stabilized metal nanoparticles for dechlorination of chlorinated hydrocarbons in soils, sediments and groundwater. PCT Int. Appl. (2007), 49 pp. CODEN: PIXXD2

WO 2007001309 A2 20070104 CAN 146:127840 AN 2007:14614 CAPLUS

Zhao, Dongye; Xu, Yinhui. In situ remediation of inorganic contaminants using stabilized zero-valent iron nanoparticles. PCT Int. Appl. (2007), 104 pp. CODEN: PIXXD2 WO 2007115189 A2 20071011 CAN 147:454365

AN 2007:1150422 CAPLUS

Wang, Qing; Li, Hong; Huang, Xuejie; Chen, Liquan. Pyrolysis-generated hard carbon material, and preparation and applications of same. PCT Int. Appl. (2001), 21 pp. CODEN: PIXXD2 WO 2001098209 A1 20011227 CAN

136:56439 AN 2001:935522 CAPLUS

U.S. Pat. No. 4,252,658, issued Feb. 24, 1981; corresponding to U.S. patent application No. 05/509,950, filed Sep. 27, 1974, Solid lubricant.

Chemical Synthesis of Carbon Microbeads Through Octatetraynes, Susan Olesik, U.S. patent pending, available at http://tlc.osu.edu/technologies/detail.cfm?TechID=239&CatID=8.

New lease of life for used cola bottles, Kathryn Wills, RSC Publishing (18 Feb. 2009), available at http://www.rsc.org/Publishing/ChemScience/Volume/2009/03/New_lease_of_life for_used_cola_bottles.asp (citing A solvent free process for the generation of strong, conducting carbon spheres by the thermal degradation of waste polyethylene terephthalate, Swati V. Pol, Vilas G. Pol, Dov Sherman and Aharon Gedanken, Green Chem., 2009, 11, 448-451).

Wang, Q.; Li, H.; Chen, L.; Huang, X., Monodispersed hard carbon spherules with uniform nanopores. Carbon 2001, 39, (14), 2211-2214.

Yang, R.; Qiu, X.; Zhang, H.; Li, J.; Zhu, W.; Wang, Z.; Huang, X.; Chen, L., Monodispersed hard carbon spherules as a catalyst support for the electrooxidation of methanol. Carbon 2005, 43, (1), 11-16.

Ponder, S. M.; Darab, J. G.; Mallouk, T. E., Remediation of Cr(VI) and Pb(II) Aqueous Solutions Using Supported, Nanoscale Zero-valent Iron, Environ. Sci. Technol. 2000, 34, 2564-2569.

Lien, H.; Zhang, W., Nanoscale iron particles for complete reduction of chlorinated ethenes, Colloids and Surfaces A: Physicochem. Eng. Aspects 191 (2001) 97-105.

U.S. Pat. No. 6,787,034, Noland, et. al.; US Patent Application Publication No. 20050006306, Noland, Scott, et. al.; and International Patent Application Publication No. WO 2009/106567 A1.

Muftikian, R.; Fernando, Q.; Korte, N., A method for the rapid dechlorination of low molecular weight chlorinated hydrocarbons in water. Water Res. 1995, 29, 2434.

Luiz C. A. Oliveiraa, Rachel. V. R. A. Riosa, Jose' D. Fabrisa, V. Gargc, Karim Sapagb, Rochel M. Lagoa, Activated carbon/iron oxide magnetic composites for the adsorption of contaminants in water. Carbon 40 (2002) 2177-2183.

The following presentations, incorporated herein by reference, were attached to our U.S. Provisional Patent Application No. 61/251,632, filed 14 Oct. 2009:

Jingjing Than, Tonghua Zheng, Bhanukiran Sunkara, Gerhard, Piringer, Gary McPherson, Yunfeng Lu, Vijay T. John, Multifunctional Colloidal and Nanoscale Materials for Targeted Remediation of Chlorinated Hydrocarbons, ACS/IACIS Symposium, Columbia University, Jun. 19, 2009.

Jingjing Zhan, Tonghua Zheng, Bhanu Sunkara, Gerhard Piringer, Gary McPherson, Yunfeng Lu, Vijay John, Reactive Composites for Targeted Remediation of TCE, Department of Chemical & Biomolecular Engineering, Tulane University. New Orleans, La.

Jingjing Zhan, Tonghua Zheng, Bhanukiran Sunkara, Gerhard Piringer, Gary McPherson, Yunfeng Lu, Vijay T. John, Multifunctional Hybrid Colloidal and Nanoscale Materials for Targeted Remediation of Chlorinated Hydrocarbons, ACS Meeting, Washington D.C. (Technology I and II).

Also incorporated herein by reference is the following:

Bhanukiran Sunkara, Jingjing Zhan, Jibao He, Gary L. McPherson, Gerhard Piringer, and Vijay T. John, Nanoscale Zerovalent Iron Supported on Uniform Carbon Microspheres for the In situ Remediation of Chlorinated Hydrocarbons, ACS Applied Material & Interfaces, available at http://pubs.acs.org/doi/abs/10.1021/am1005282.

### BRIEF SUMMARY OF THE INVENTION

Initial results indicate that these carbon particles are microporous with surface areas on the order of 400 m²/g. They adsorb TCE to the same extent as activated carbons as shown in FIG. 10 which illustrates a preliminary adsorption experiment done using 20 mL of 20 ppm TCE solution containing 0.2 g of the particles. The invention encompasses the impregnation of these carbon microspheres with ZVI particles and stabilizes these composites in solution using polymers (e.g., carboxymethyl cellulose).

Zerovalent iron nanoparticles have significant uses in environmental remediation. They are capable of breaking down dense non aqueous phase liquids, typically chlorinated hydrocarbons such as trichloroethylene. These compounds are amongst the most recalcitrant of pollutants and are hard to reach since they penetrate below the water table and permeate through aquifers. The difficulty with using zerovalent iron is that these particles are magnetic and stick to each other. It is very difficult to get these particles to penetrate through sediments.

We have developed new technology using nanocarriers of carbon. Carbon is environmentally benign, and furthermore has very interesting properties of being able to adsorb chlorinated hydrocarbons. We have been able to synthesize 50 nm to 6 μm (preferably 200 nm to 6 μm; more preferably 200 nm to 1.5 μm; even more preferably 300-700 nm; most preferably 400-600 nm; e.g. 500 nm) sized carbon spheres using a hydrothermal synthesis technique. The precursors are sugars or complex carbohydrates. This is by itself, not new. Sugars such as sucrose have been used to make carbon spheres. The novelty in our work, however, is the ability to introduce zerovalent iron nanoparticles onto these spheres and to use these spheres for environmental remediation of chlorinated hydrocarbons. The carbon spheres are of the optimal size for transport through sediments. This is an application that, to the best of our knowledge, has not been disclosed in the patent or journal literature.

The methodology may also be used in the remediation of arsenic. Chlorinated hydrocarbons are different from arsenic. When remediating arsenic, iron oxide is used in place of the zerovalent iron particles. The same carbons spheres are preferably used. Arsenic occurs naturally in soil, and in higher concentrations in some soils than others. It is especially found in places where it can get into the well water and the water requires filtration. Also, the particles of the present invention can be used in situations where there are multiple contaminants, such as chlorinated hydrocarbons and arsenic (with perhaps a mixture of zerovalent iron particles and iron oxide on the carbon particles). The zerovalent iron particles come in together with the carbon microspheres only because the chlorinated hydrocarbons go deep down into the soil and earth and slowly come out in lakes, etc. The remediation of arsenic is for drinking purposes, thus the use of iron oxide instead to have control and immobilize the arsenic, not to break it down.

As used herein, monodisperse means all particles having the same size+−10%. It might be helpful to adjust or tune the size of the particles depending upon the soil conditions, and the optimum size might be discovered through experimentation. At times, it might be helpful to have all particles be about the same size, or within 10% of the same size, or within 50% of the same size. At times, bidispersity or polydispersty of particles might be desired (such as for example when soil is not homogenous or particles less expensive if polydisperse).

Thus the technology involves loading iron nanoparticles onto these carbon microspheres and using the carbon microspheres to transport the iron to the sites of contamination. The carbon microspheres may also be utilized in reactive barrier technology.

The present invention also includes an aerosol-based method to prepare efficient carbon supported zerovalent iron particles for environmental remediation of chlorinated hydrocarbons. Spherical iron-carbon nanocomposites were developed through a facile aerosol-based process with sucrose and iron chloride as starting materials. These composites exhibit multiple functionalities relevant to the in situ remediation of chlorinated hydrocarbons such as TCE. The distribution and immobilization of iron nanoparticles on the surface of carbon spheres prevents zerovalent nanoiron aggregation with maintenance of reactivity. The aerosol-based carbon spheres allow adsorption of TCE, thus removing dissolved TCE rapidly and facilitating reaction by increasing the local concentration of TCE in the vicinity of iron nanoparticles. The strongly adsorptive property of the composites also prevents release of any toxic chlorinated intermediate products. The nanoscale composite is in the optimal range for transport through groundwater saturated sediments. Furthermore, these iron-carbon composites can be designed at low cost and the materials are environmentally benign.

We have also demonstrated that spherical particles of different sizes (i.e., ranging from the nanometer to micrometer lengthscale), composition, and surface coating (e.g., surfactants, polymers, proteins) are effective oil and water-based lubricants, which lower the friction forces between shearing surfaces, even at relatively high loads.

### BRIEF DESCRIPTION OF THE SEVERAL VIEWS OF THE DRAWINGS

For a further understanding of the nature, objects, and advantages of the present invention, reference should be had to the following detailed description, read in conjunction with the following drawings, wherein like reference numerals denote like elements and wherein:

FIG. 1 shows the design of functional composite particles for effective transport, reaction and partitioning.

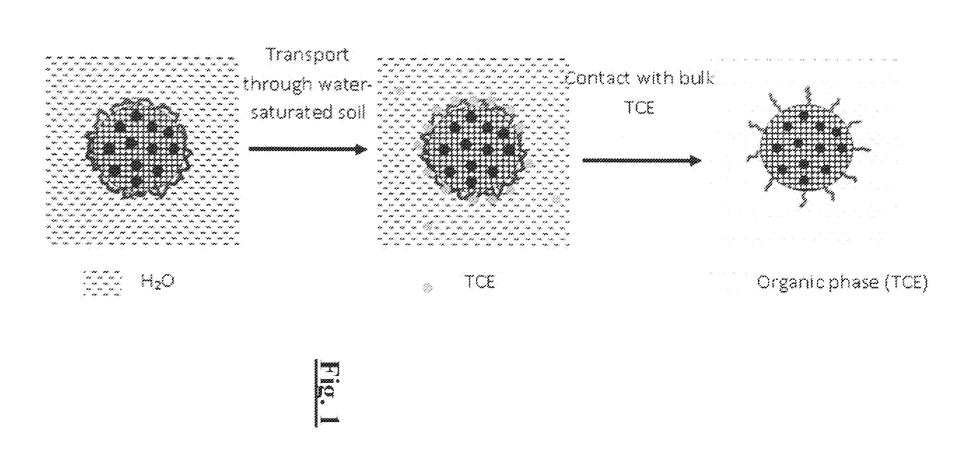

FIG. 2 illustrates the encapsulation of NZVI in porous silica. The silica precursors are shown at the top, the aerosolizer in the middle and the “chemistry in a droplet” concept at the bottom.

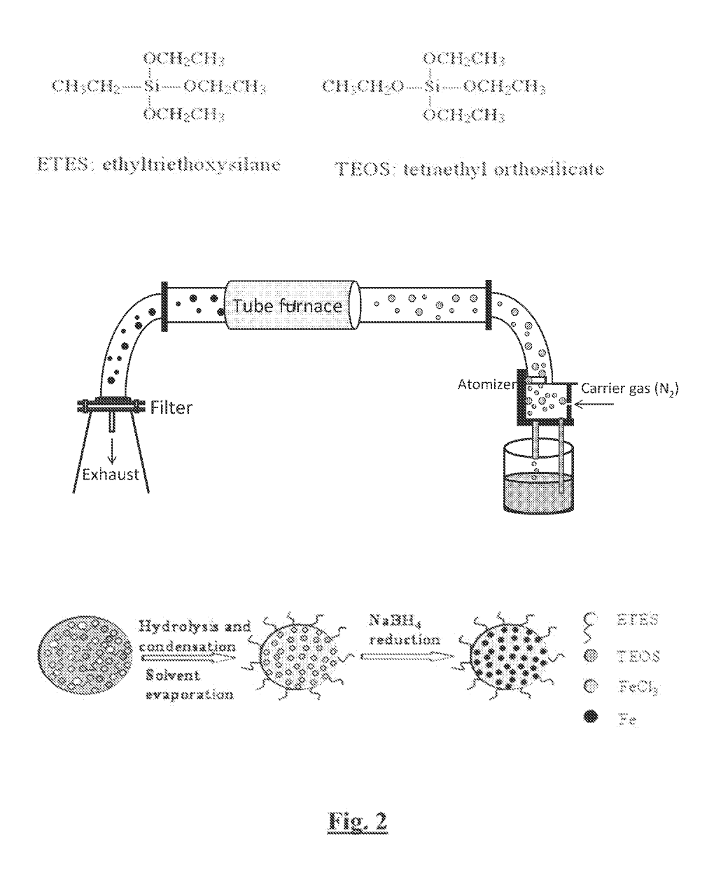

FIG. 3 shows the Size Distribution of Fe/Ethyl-Silica Particles. The inset shows a TEM of the particles.

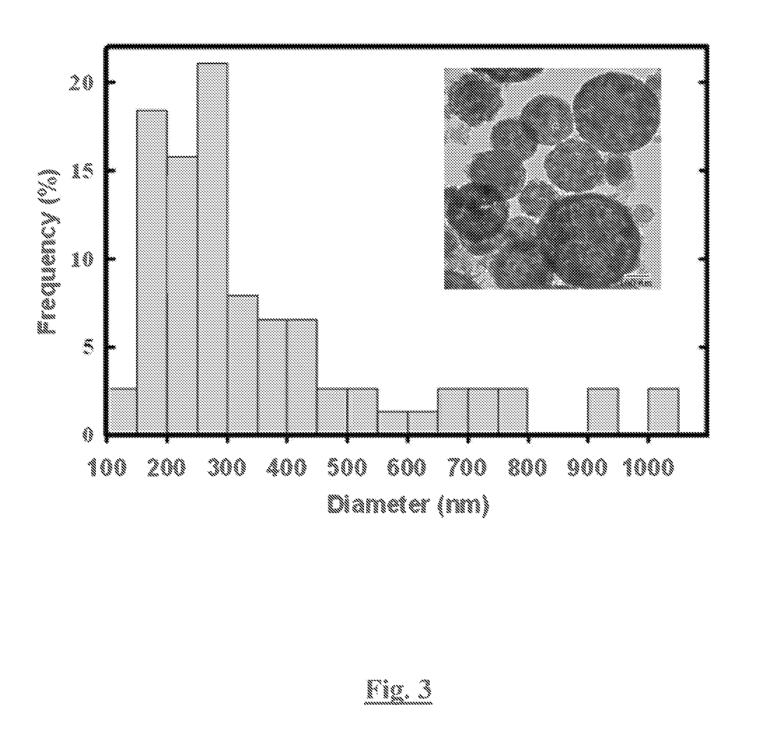

FIG. 4 illustrates Reactivity Characteristics of the composite Fe/EthylSilica particles. The initial drop in solution TCE concentration is due to adsorption, bringing up the fact that these are adsorptive-reactive particles.

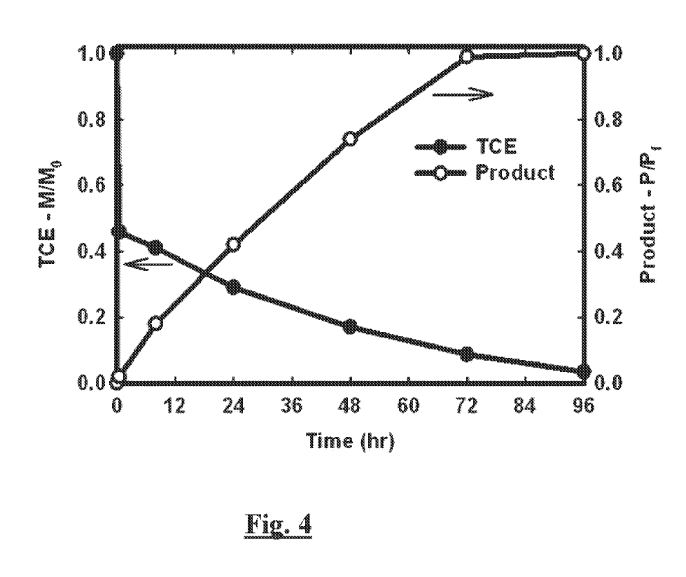

FIG. 5 shows the TE equation applied to Fe-based systems. Inset (a) is the commercial system of bare RNIP which aggregates, inset (b) is the Fe/Ethyl-Silica system.

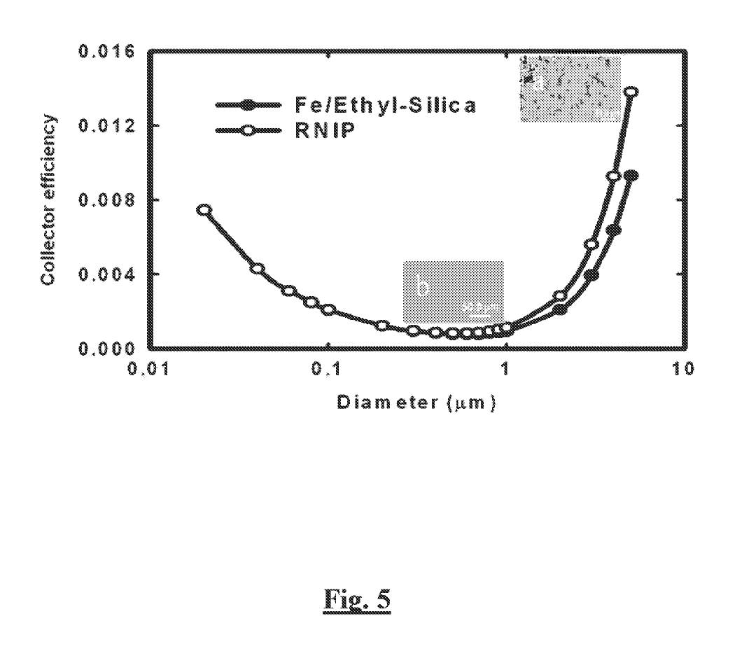

FIG. 6 illustrates the Column elution profiles of Fe/Ethyl-Silica particles compared to bare RNIP.

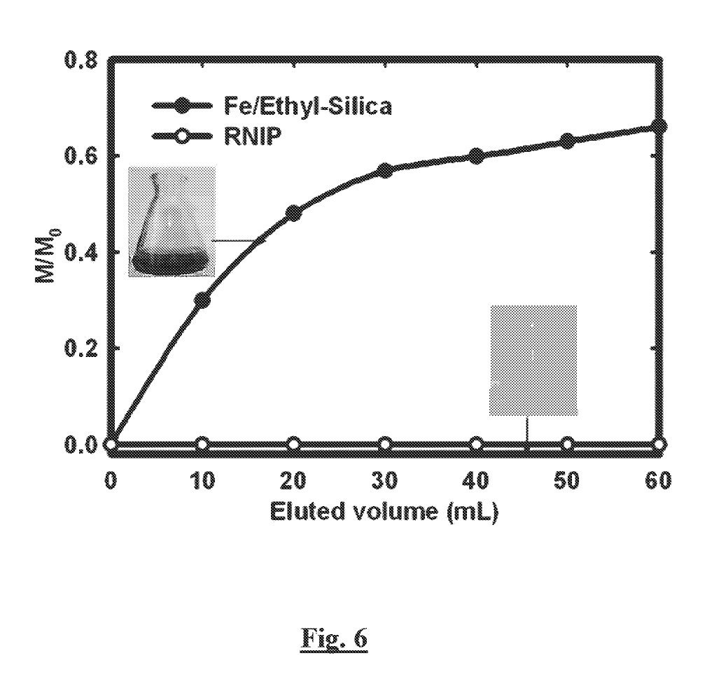

FIG. 7 illustrates capillary transport studies. The schematic of the setup is at the top, the middle panels illustrate the packed capillaries at varying stages of elution, and the bottom panels indicate the micrographs of the capillaries.

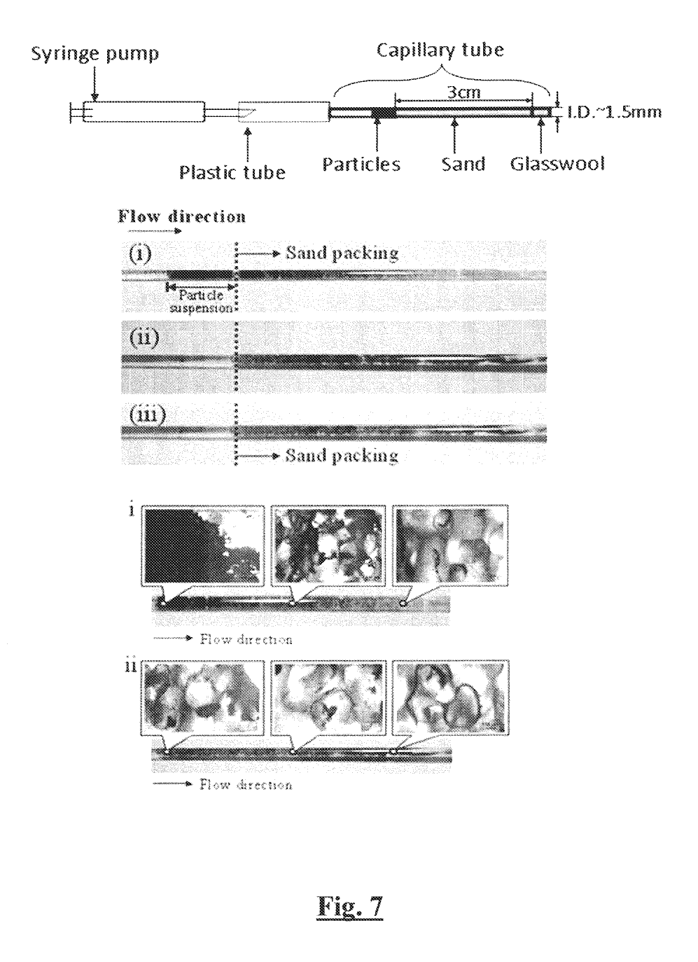

FIG. 8 illustrates a microcapillary visualization experiment where a TCE droplet is injected using a micropipetter into a 200 μm capillary containing dispersed Fe/Ethyl-Silica particles in water. We see a stable aggregation of the particles on the TCE droplet interface.

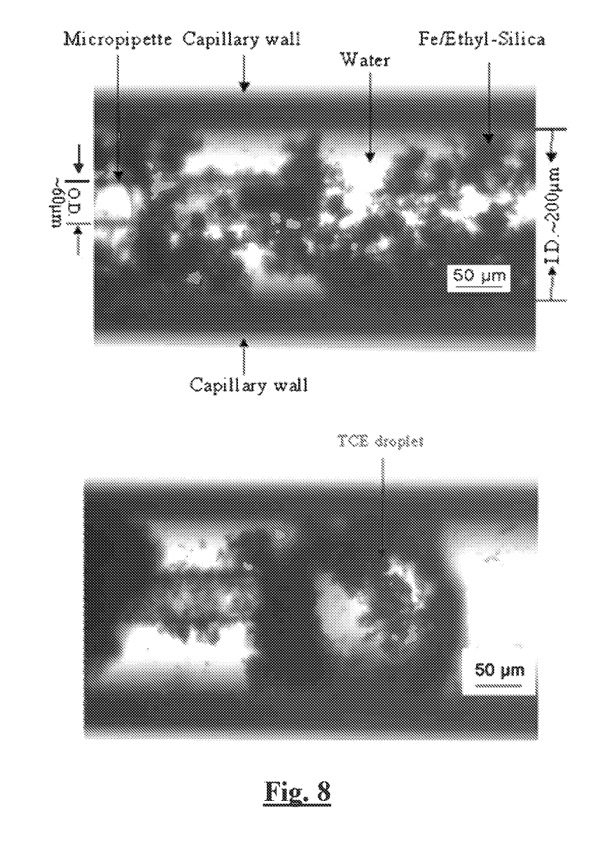

FIG. 9 illustrates the synthesis of monodisperse carbon particles.

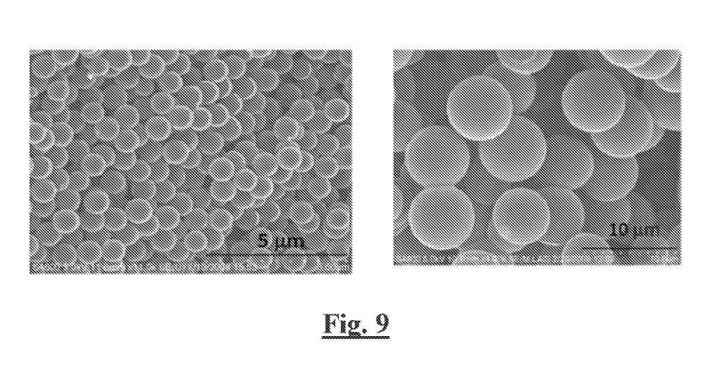

FIG. 10 illustrates adsorption capacities of porous carbon microspheres. Y is the percent of TCE adsorbed.

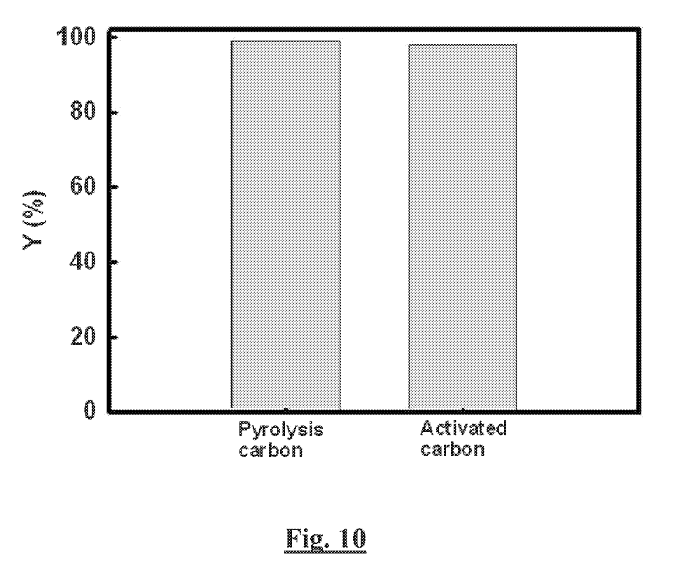

FIG. 11 illustrates (a) SEM; (b) TEM of 500 nm carbon particles obtained from hydrothermal dehydration and pyrolysis of sucrose; (c) Schematic of the multifunctional particulate system showing a carbon particle with physisorbed CMC containing NZVI. The dots signify TCE in solution and adsorbed on the carbon.

FIG. 12 illustrates TCE removal from solution and gas product evolution rates for (a) CMC+Fe+carbon (System I); (b) CMC stabilized Fe nanocolloids (System II); and (c) (CMC+Fe)/carbon (System III) without unadsobed Fe and excess CMC. M/M₀ is the fraction of the original TCE remaining and P/P_{f} is the ratio of the gas product peak to the gas product peak at the end of 100 minutes. In all cases, the amount of NZVI was kept constant at 0.02 g in 20 mL of 20 ppm TCE solution.

FIG. 13 illustrates comparison of adsorption capacities of CMC, CMC+carbon microspheres, pristine carbon microspheres, and commercial activated carbon. In all experiments, 20 mL of a 20 ppm TCE solution was used. Other component levels were 0.16 g CMC, 0.1 g carbon microspheres, or 0.1 g activated carbon.

FIG. 14 illustrates (a) Stability of CMC+NZVI and CMC+NZVI+carbon systems in water; (b) Partitioning characteristics of CMC+NZVI and CMC+NZVI+carbon when contacted with a two-phase water-TCE system.

FIG. 15 illustrates characterization of transport through packed capillaries (a) experimental set-up. Flow rate: 0.1 mL/min, sand length: 3 cm and injected suspension volume: 0.03 mL; Photograph of capillary (b) before, (c) during and (d) after water flushing. Panel i-iii showing optical micrographs of sediments and particles at various locations after water flushing (all scale bars are 50 μm). Panel iii illustrates accumulation on glass wool at the end of the capillary.

FIG. 16 illustrates (a) TEM of (CMC+NZVI)/carbon particles (b) higher resolution TEM image of a single particle showing the distribution of NZVI. (c) SEM of (CMC+NZVI)/carbon particles.

FIG. 17 illustrates morphology of CMC+NZVI+carbon (a) before passage through the capillary (b) after passage through the capillary.

FIG. 18 illustrates the aerosol reactor.

FIG. 19 illustrates once the particles are collected they are dispersed in aqueous solution to which we add sodium borohydride to reduce the iron oxides and iron hydroxides to zerovalent iron. The resulting particles are shown.

FIG. 20 illustrates (a) Structure of sodium carboxymethylcellulose (CMC) (b) SEM of 500 nm carbon particles obtained from hydrothermal dehydration and pyrolysis of sucrose. (c) Schematic of the multifunctional particulate system showing a carbon particle with physisorbed CMC containing NZVI. The red dots signify TCE in solution and adsorbedon the carbon.

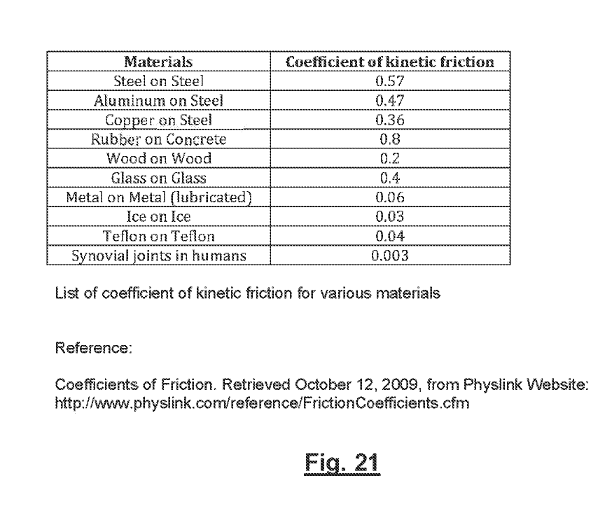

FIGS. 21 and 22 illustrate experimental data with characterization of the lubrication properties including plots of the coefficient of friction vs. load. FIG. 21 was retrieved on Oct. 12, 2009, from Physlink Website: http://www.physlink.com/reference/FrictionCoefficients.cfm. FIG. 22 illustrates data for the effectiveness of using hard carbon spheres (HCS) as lubricants.

FIG. 23 shows how the aerosol process is done.

FIG. 24 is a scanning electron micrograph (SEM) of the carbon particles covered with the iron.

FIG. 25 are transmission electron micrographs TEM (a-e) and cut-section TEM (f) of Fe⁰-C particles show NZVI is supported on the carbon surface.

FIG. 26 shows the extremely rapid rate of destruction of TCE.

FIG. 27 is a gas chromatographic trace of the fast reaction.

FIG. 28 shows that the carbon (+iron) particles are stabilized by the addition of CMC.

FIG. 29 shows SEMs of the particles made through the aerosol process at different temperatures.

FIG. 30 shows TEMs of the particles made through the aerosol process at different temperatures.

FIG. 31 shows cut section TEMs of the particles made through the aerosol process at different temperatures.

FIG. 32 shows a schematic of the carbothermal reduction apparatus.

FIG. 33 shows (a) Schematic of aerosol reactor for composite synthesis and (b) schematic of reaction in an aerosol droplet.

FIG. 34 shows (a) TEM of carbon prepared by an aerosol-based process; (b) TEM, (c) cut-section TEM and (d) SEM of Fe/C. The inset is the low magnification TEM of Fe/C.

FIG. 35 shows TCE removal from solution and gas product evolution rates for Fe/C composites. M/M0 is the fraction of the original TCE remaining and P/Pf is the ratio of the gas product peak to the gas product peak at the end of 8 hours.

FIG. 36 shows representative GC trace of headspace analyses showing TCE degradation and reaction product evolution at various reaction time.

FIG. 37 shows comparison of adsorption capacity of humic acid, Fe/C from an aerosol-based process (1000° C.) and commercial activated carbon. In all experiments, 20 mL of a 20 ppm TCE solution, 0.2 g of particles were used.

FIG. 38 shows sedimentation curves of Fe/C composites in 4% (w/w) CMC solution (solid circles) and water (open circles). The inset images are Fe/C composites in CMC solution and water after 24 h, respectively.

FIG. 39 shows experimental set-up to study transport in horizontal capillaries. Photographs of before (top) and after (bottom) water flushing showing the characteristics of transport through packed capillaries. Panels showing optical micrographs of particles at various locations (i) in the middle of the capillary and (ii) on glass wool at the end of the capillary after water flushing (all scale bars are 100 μm). Flow rate: 0.1 mL/min, sand length: 3 cm and injected suspension volume: 30 μL.

### DETAILED DESCRIPTION OF THE INVENTION

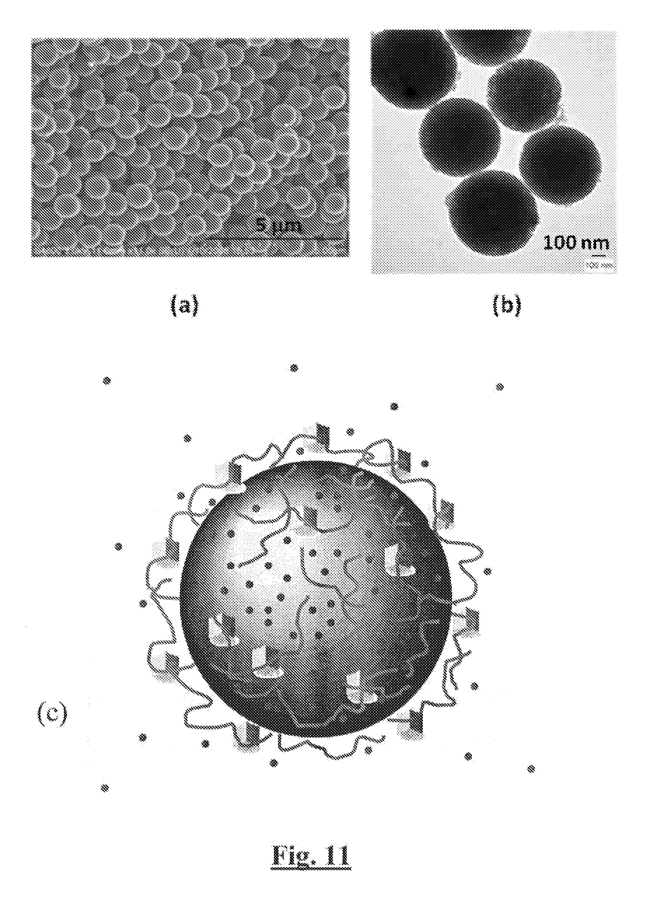

Some of the concepts behind an embodiment of the present invention are illustrated in FIG. 11c which shows a schematic of a carbon microsphere decorated with CMC embedded with NZVI. Our objective is to couple the use of CMC with the carbon microspheres and use the polymer to prevent NZVI from aggregation and maintain solution stability of the carbon colloids. The use of CMC as an anionic polyelectrolyte to enhance colloid stability is established and its ability to adsorb onto hydrophobic surfaces has been well-characterized [32A-34A]. In a close analogy, CMC has been used as a dispersant for coal-water slurries in a recent study [35A] indicating its potential applicability to disperse carbon microspheres. In the current application, the following characteristics are expected to be applicable (1) the NZVI supported on CMC are expected to maintain activity to the dechlorination of TCE (2) in analogy with the adsorptive properties of activated carbon, the carbon microspheres are expected to strongly adsorb TCE thereby potentially reducing solution TCE content (3) the size and monodispersity of the carbon microspheres may facilitate optimal transport of these particles in groundwater. In addition, we hypothesize that these carbon particles would easily partition to TCE bulk phases and in so doing, would pull the corona of polymer and NZVI also into the TCE bulk phase. Finally, all these materials are easily available, inexpensive and environmentally benign, and the solution synthesis of the carbon microspheres would indicate scalability to manufacturing volumes. We note that similar concepts of CMC stabilizing ZVI-Carbon for enhanced transport in the remediation of hexavalent chromium, and the possible use of carbon as an adsorbent have been described in the literature by Mallouk and coworkers [17A, 37A]. The novelty of our approach is the use of highly monodisperse carbons to control transport and colloidal stability, and the exploitation of coupled reaction and adsorption, with the ZVI particles attached to the polymer.

Functionalizing with a polymer usually refers to chemically attaching a polymer to a particle. The particle then has modified properties of stability since it has a coating of the polymer. The polymer can be a polyelectrolyte. For example an anionic polyelectrolyte will be negatively charged and will repel other particles functionalized with the same polymer. Hence the two particles will not form an aggregate and can stay suspended in solution if the particle is small enough.

Instead of chemically attaching a polymer to the particle, an easier way is to simply physically adsorb the polymer to the particle. While the binding is not as strong, the polymer may be able to wrap around the particle and form a loose coating. This is the simple way of stabilizing with a polyelectrolyte and this is what we prefer to do.

### Experimental Section

Chemicals.

Chemicals including sucrose (ACS reagent), sodium carboxymethyl cellulose (NaCMC or CMC, mean MW=90 000, low viscosity), sodium borohydride (NaBH₄, 99%), potassium hexachloro-palladate (IV) (K₂PdCl₆, 99%) and trichloroethylene (TCE, 99%) were purchased from Sigma-Aldrich. Ferrous sulfate heptahydrate (FeSO₄.7H₂O, certified ACS reagent) was from Fisher Scientific. All chemicals were used as received without any further treatment. Deionized (DI) water generated with a Barnstead E-pure purifier (IA) to a resistance of approximately 18 MS2 was used in all experiments.

Preparation of CMC stabilized NZVI+Carbon Colloidal Particles.

The preparation of the carbon support includes two steps (a) a hydrothermal dehydration step and (b) a pyrolysis (carbonization) treatment. The process is similar to that reported in the literature [29A, 30A] but with minor modifications, and is briefly described. 45 mL of 0.15 M sucrose water solution was introduced into a 50 mL stainless steel autoclave vessel, which was then closed with a stainless steel cap. The vessel was heated at 190° C. for 5 hours subjecting the sucrose to hydrothermal treatment. The resulting solids suspension was centrifuged and washed three times with ethanol. The collected particles were placed in the hood to air-dry overnight. In the subsequent pyrolysis step, the dry particles were placed in a tube furnace, which was held at 1000° C. for 5 h under flowing argon. The obtained carbon particles were stored in an airtight vial. BET surface areas of the carbon microspheres were measured at 320 m²/g.

The preparation of CMC stabilized NZVI+carbon colloidal particles was based on the method of preparing CMC stabilized nanoscale zerovalent iron particles as described by He and coworkers [18A, 36A] with modifications to accommodate the additional carbon component. 100 mL of 0.96% (w/w) CMC aqueous solution combined with 10 mL of freshly prepared 0.21 M FeSO₄.7H₂O solution was stirred for 15 minutes in a N₂ atmosphere, allowing the formation of the Fe²⁺-CMC complex. While maintaining inert conditions, the sample was transferred to an Erlenmeyer flask and 10 mL of 0.42 M sodium borohydride solution was added drop-wise followed by the addition of 0.6 g of as-prepared carbon particles in one aliquot. When hydrogen evolution ceased, the sealed flask was placed on a rotary shaker at 60 rpm for 2 hours to facilitate adsorption of CMC and NZVI onto the carbon surface. The zerovalent iron particles were then loaded with catalyst Pd by adding 100 μL of 0.0057 M K₂PdCl₆ to the suspension. Accordingly, the final composition of CMC stabilized NZVI+carbon colloidal particles used in this study is 0.8% (w/w) CMC, 1 g/L NZVI, 0.05% Pd (w/w of NZVI) and 5 g/L carbon.

To separate carbon supported NZVI particles from unadsorbed CMC+NZVI, the suspension was centrifuged using an Eppendorf Centrifuge 5800 at 4000 rpm for 10 minutes to precipitate the carbon and attached CMC+NZVI. The iron content of the supernatant was analyzed by complexation with 1,10-phenanthroline followed by absorbance measurement of [Fe(phen)₃]²⁺ at 508 nm[37A, 38A]. In the experiments reported herein 40% of the NZVI is precipitated with carbon with the remaining NZVI attached to unadsorbed CMC. The fraction of NZVI+CMC attached to carbon can be easily increased by the addition of carbon. For example, when the amount of carbon is doubled to 10 g/L, over 70% of the NZVI+CMC becomes attached to the carbon. It is possible to get a lot more than 70 percent by increasing the carbon. Alternate routes exist to enhance the adsorption of CMC to carbon. CMC is added to the carbon particles and the material is centrifuged to precipitate the carbon and attached CMC+NZVI. The remaining supernatant is evaporated off to force the CMC in solution to adsorb to the carbons. If such methods are used, the fraction of CMC+NZVI adsorbed to carbon becomes close to unity.

As a reference sample, we have also prepared CMC stabilized zerovalent iron without the addition of carbon, using the method of He and coworkers [18A, 28A, 36A].

Characterization.

Transmission electron microscopy (TEM, JEOL 2010, operated at 120 kV voltage) and field emission scanning electron microscopy (SEM, Hitachi S-4800, operated at 20 kV) were used to characterize the morphology of the particles. Optical microscopy (Olympus IX71, Japan) was used to analyze the fate of the particles in porous media. A Malvern Nanosizer (Southborough, Mass.) was used to measure surface charge density through the ξ-Potential.

Analytical.

TCE dechlorination effectiveness was tested in a series of duplicated batch experiments. In all tests, the concentrations of NZVI and TCE were maintained at 1 g/L and 20 ppm. In detail, 20 mL of freshly prepared CMC+NZVI/carbon or CMC+NZVI colloidal particles were added to a 40 mL vial capped with a Mininert valve. TCE degradation was initiated by spiking 20 μL of a TCE stock solution (20 g/L TCE in methanol) into the solution containing the nanoparticles, which resulted in an initial TCE concentration of 20 ppm. The reaction was monitored through headspace analysis using a HP 6890 gas chromatography (GC) equipped with a J&W Scientific capillary column (30 m×0.32 mm) and flame ionization detector (FID). Samples were injected splitless at 220° C. The oven temperature was held at 75° C. for 2 min, ramped to 150° C. at a rate of 25° C./min and finally held at 150° C. for 10 min to ensure adequate peak separation between TCE, chlorinated and non-chlorinated reaction products.

### Results and Discussion

### Adsorption and Reactivity Studies

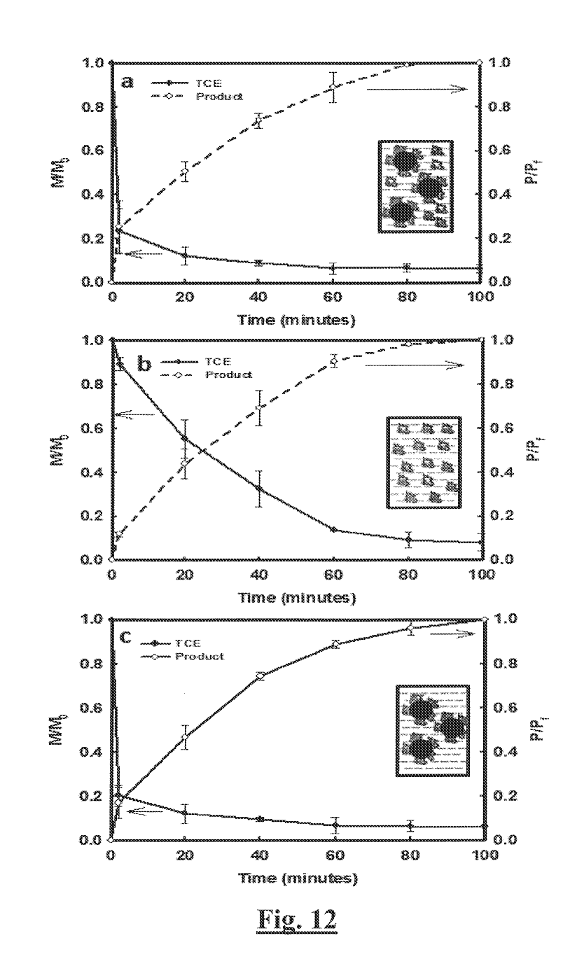

FIG. 12 illustrates reactivity characteristics of iron containing colloidal systems when contacted with dissolved TCE. There are three cases that we have considered in order to understand the reactivity of these systems. In the first case (FIG. 12a), the reactivity of the entire system containing both CMC+NZVI attached to the carbon, and free CMC+NZVI, is measured—we denote this as CMC+NZVI+carbon (System I). The second case considered (FIG. 12b) is the control where the reactivity of a carbon-free system, CMC+NZVI (System II), is measured. The sample in the third case (FIG. 12c) represents the situation where only CMC+NZVI attached to carbon is considered. This sample was obtained by centrifuging the sample in case I wherein CMC+NZVI strongly adsorbed to carbon precipitates out and free CMC+NZVI remains in the supernatant. We denote this system without free CMC+NZVI as (CMC+NZVI)/carbon (System III) to characterize the carbon support. In all three cases, the NZVI (and Pd) content has been kept constant at 20 mg of NZVI in 20 mL of solution. To keep the NZVI (and Pd) content constant, System III involves a proportionally increased level of (CMC+NZVI)/carbon; in this case a 2.5 fold increase in carbon since 40% of the CMC+NZVI in system I is adsorbed on carbon.

Clear observations are immediately apparent FIG. 12. The samples with carbon indicate a very sharp initial decrease in solution TCE concentration. This is clearly not due to reaction but due to rapid adsorption of TCE onto the carbon microspheres. At these levels of carbon addition and initial solution TCE concentration, within experimental error, almost all the solution phase TCE becomes adsorbed onto the carbon. The evolution of gas phase products is significantly slower than the drop in TCE solution concentration, further indicating that reaction is the slow step in the combined adsorption+reaction sequence. If we therefore assume that reaction is rate controlling, it is possible to calculate a pseudo-first order rate constant by following the lumped gas phase products (B) in the reaction A→B, and relating this to the loss of TCE (reactant A). The first order rate constant is approximately 2.1 h⁻¹ in all three cases, as the product evolution data are not noticeably different, indicating that the ZVI is equally accessible to TCE whether the TCE is in free solution or is adsorbed onto the carbon. The reaction rate is strongly dependent on the catalytic role of Pd involving dissociative chemisorption of H₂. In accordance with the study by Lien and Zhang [39A], we have also observed that in the absence of Pd, the degradation rate of TCE drops by over two orders of magnitude. Clearly, the results of FIG. 12 indicate no inhibitory aspect in the reaction rate upon TCE adsorption and we can thus consider the adsorption/desorption step of TCE as being in equilibrium with the overall rate controlled by the surface reaction associated with TCE dechlorination over the NZVI and Pd complex [39A]. We consider that the adsorptive-reactive system proposed here is well-suited for TCE remediation as it also provides a strong sequestration mechanism in addition to reactive decontamination.

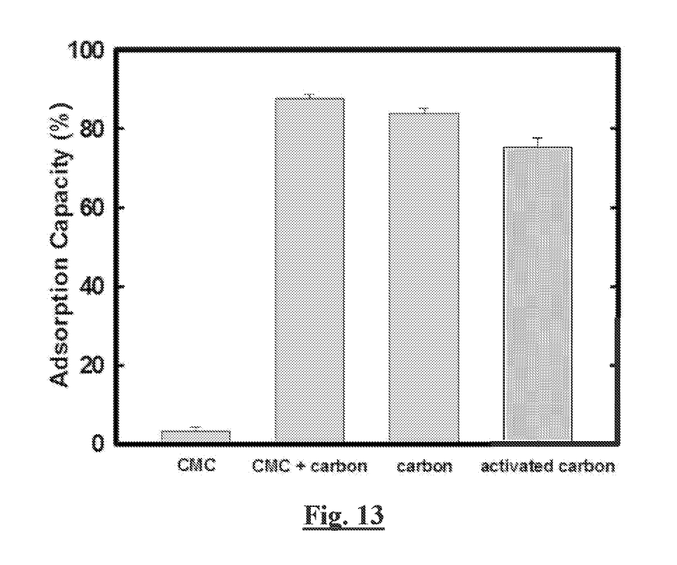

Further characterization of the adsorptive properties involving the carbon microspheres is shown in FIG. 13. In all experiments, 20 mL of a 20 ppm TCE solution was used. The CMC added to this solution was 0.16 g, and the carbon levels either as microspheres or as granular activated carbon was 0.1 g. Typically we go from a weight ratio of CMC to Carbon of 1:1 to 100:1. In other words, if the carbon mass is 50 mg/L, the 1:1 ratio gives CMC as 50 mg/L and the 100:1 ratio gives CMC as 5000 mg/L. The more CMC, the greater the solution stability. Clearly, the adsorption of TCE on CMC is extremely negligible in comparison with its adsorption on the carbon microspheres, and the presence of CMC does not inhibit access or adsorption to the carbon microspheres. The CMC+carbon microspheres adsorption is a little higher than that of pristine carbon microspheres due to the additional presence of CMC. Finally, the level of adsorption on carbon microspheres is comparable to that on commercially available granular and irregularly defined activated carbons.

We have also calculated the partition coefficient for TCE adsorption on the carbon microspheres using the comprehensive definition of Phenrat and coworkers [40A]

$$\frac{{C}_{TCE}^{ads}}{{C}_{TCE}^{water}} = {K}_{p} = \{ \frac{[ \begin{matrix}{( {C}_{TCE}^{Air} )}_{ref} {V}_{hs} - \\ {( {C}_{TCE}^{Air} )}_{ads} {V}_{hs}\end{matrix} ] + [ {( \frac{{C}_{TCE}^{Air}}{{K}_{H}^{TCE}} {V}_{water} )}_{ref} - {( \frac{{C}_{TCE}^{Air}}{{K}_{H}^{TCE}} {V}_{water} )}_{ads} ]}{( \frac{{m}_{p}}{{ρ}_{p}} ) {( \frac{{C}_{TCE}^{Air}}{{K}_{H}^{TCE}} )}_{ads}} \}$$

Where C_{TCE}^{ads} is the concentration of TCE on the adsorbent (mol/L), C_{TCE}^{water} is the concentration of TCE in the water phase (mol/L), C_{TCE}^{Atr} is the concentration of TCE in the headspace (mol/L), Vₕₛ and V_{water} are the volumes of the headspace and water, respectively (L), M_{ads} is the mass of the adsorbent (g), Pads is the density of the adsorbent (g/L). The subscripts ref and ads refer to the system without and with the adsorbent. K_{H}^{TCE*} is the Henry's law constant for TCE partitioning in water, with a value of 0.343 at 25° C. [8A]. The measured partition coefficient for TCE adsorption on CMC is 14.5, in close agreement with that measured by Phenrat and coworkers [40A]. On the other hand, Kₚ for the adsorption of TCE on carbon is 3913 constituting an almost 300 fold increase in adsorption capacity.

### Stability and Partitioning Characteristics

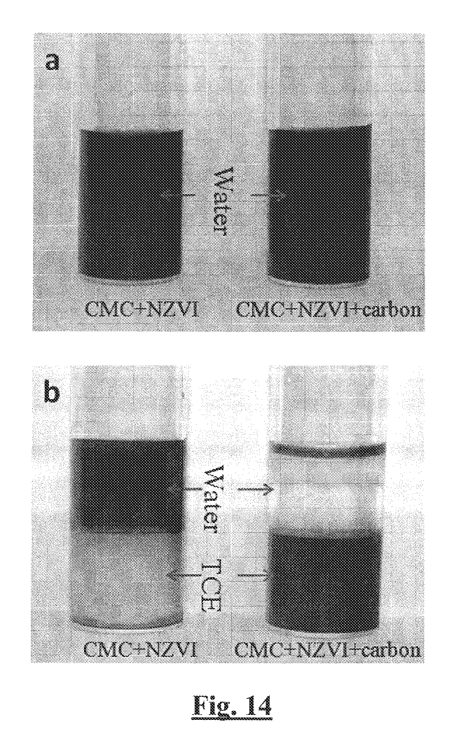

The colloidal stability of NZVI based systems is a key factor in assessing transportability in groundwater [41A]. FIG. 14 illustrates simple visual studies of suspension and partitioning characteristics of the carbon based systems. The samples were probe sonicated to enhance mixing and allowed to equilibrate. FIG. 14a illustrates suspension stability of samples in water and it is clear that CMC stabilizes the carbon particles. All suspensions are indefinitely stable in water (>3 days) and the stabilizing effect of CMC as an effective colloid dispersant [18A, 28A, 42A] is demonstrated. FIG. 14b illustrates a remarkable aspect of introducing carbon to the system (System I in FIG. 12) when a bulk TCE phase is in contact with a bulk aqueous phase. On the left, the system with CMC+NZVI retains suspension stability in the water phase. However, on the right, we see that the system entirely partitions to the TCE phase and the water-TCE interface (a denser layer is seen at the interface at close inspection). The results indicate the ability of the system to partition to bulk TCE due to the tendency of the hydrophobic carbon to partition to the organic phase. The addition of carbon therefore serves both to sequester dissolved TCE upon transport through water, and to partition to the TCE phase upon reaching bulk TCE, thereby being stabilized in a bulk TCE phase. We also postulate that the hydrophilic CMC is hydrated upon being carried into the TCE phase thereby making water easily available to the NZVI+Pd complex facilitating hydrogen production. The combined CMC+carbon system may function analogous to a surfactant micelle with the carbon serving as a solid hydrophobic core and the CMC as the hydrophilic shell.

The role of CMC in stabilizing carbon is also shown through 4-potential measurements. In measuring the ξ-potential, solutions containing 0.8 wt % CMC (8 g/L) and 25 mg/L carbon were made with varying NaCl concentrations to provide information over a range of groundwater electrolyte concentrations. Table 1 below lists the ξ-potential of these systems. Based on broad ξ-potential classifications [43A], the values for bare carbon of around −6.3 mV indicates a system that is not colloidally stable, while the values for CMC stabilized carbon indicates systems that are stable over the electrolyte concentrations studied. Visual observations indicate that the bare carbon particles settle out over a period of 2-3 hours, while the CMC stabilized particles remain stable in solution with an extremely slow sedimentation observed after more than 3 days.

TABLE 1
Comparison of ξ-potentials for CMC + carbon systems at varying
electrolyte concentrations.

| Sample | Zeta Potential (mV) |
|---|---|
| Carbon | −6.3 ± 4.9 |
| CMC + carbon | −54.8 ± 3.6 |
| (No salt) | |
| CMC + carbon | −41.8 ± 4.9 |
| (1 mM NaCl) | |
| CMC + carbon | −35.6 ± 5.3 |
| (10 mM NaCl) | |
| CMC + carbon | −24.3 ± 4.2 |
| (100 mM NaCl) | |

### Transport Characteristics

Filtration theory predicts that the migration of colloidal particles through porous media such as soil is typically dictated by Brownian diffusion, interception and gravitational sedimentation [44A]. The Tufenkji-Elimelech (T-E) model is perhaps the most comprehensive model to describe these effects in the presence of interparticle interactions [45A] through a quantity, η₀, the collector efficiency, simply defined as the ability of the sediment to collect migrating particles, thus limiting transport through the subsurface. Optimal mobility through the sediment is when the collector efficiency is at a minimum which typically occurs at a broad particle size range from about 0.1 μm to 1 μm depending on the particle physical properties and groundwater flow characteristics [17A, 23A, 45A]. Extremely small particles do not easily transport through the soil since they do not easily follow flow streamlines as Brownian motion leads to frequent collisions with sediment grains, while large particles sediment and are filtered out. The 500 nm to 5 μm (around 50 nm to 6 μm; preferably 200 nm to 6 μm; more preferably 200 nm to 1.5 μm; even more preferably 300-700 nm; most preferably 400-600 nm; e.g., 500 nm) size range of the carbon particles indicate optimal mobility through the T-E equation. With a corona of adsorbed polymer, the effective size is somewhat larger, but still well within the optimal range of collector efficiency values.

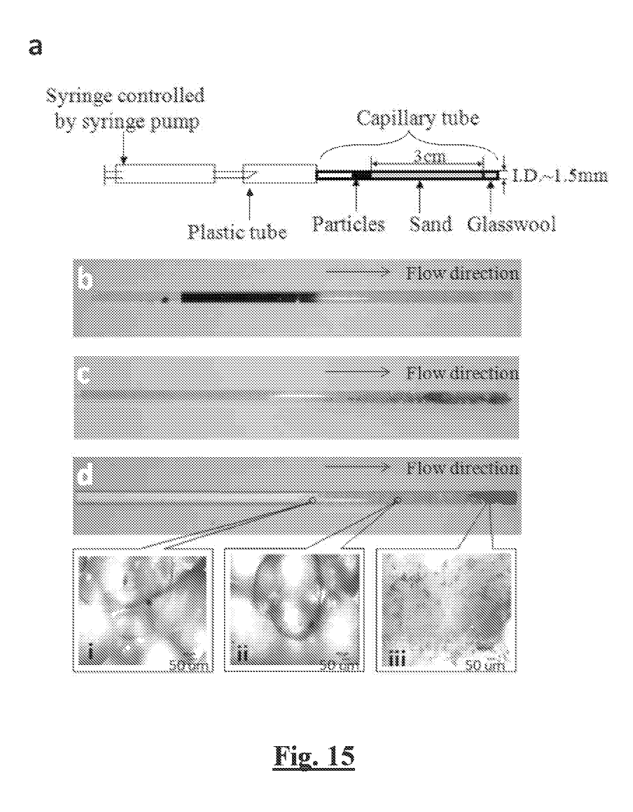

Capillary transport experiments on the CMC+NZVI+carbon system were carried out to transport characteristics of this system. This is a simple and intuitive method to study particle transport through porous media, and has been described in our previous work [23A]. Briefly, glass melting-point tubes with both ends open (1.5-1.8 mm i.d.×100 mm length, Corning, N.Y.) were used as capillaries. The capillary tubes were packed with wet Ottawa sand over a 3 cm length and were placed horizontally to simulate groundwater flow. A continuous water flow at a flow rate of 0.1 mL/min (Darcy velocity: 5 cm/min) was provided by a syringe pump. The exit point of the capillary was capped with a small glass wool plug. After 30 μL of CMC+NZVI+carbon suspension was injected into the inlet of the capillary, water flushing was initiated and an inverted optical microscope was used to observe the pore-scale transport of the particles. FIG. 15 illustrates photographs of the capillaries depicting the capillary containing CMC+NZVI+carbon colloids before, during, and after the water flush. The images indicate that carbon supported NZVI particles readily transport through the packed capillaries and become captured in the glass wool. In contrast, our earlier work has demonstrated that bare NZVI particles agglomerate and do not transport through the capillary [23A].

In addition to the collector efficiency concept described by the T-E model, bridging and attachment between the particles and the surfaces of the soil grains influence transport. Such phenomena is typically described by the sticking coefficient (a), which is primarily affected by electrostatic interactions between carrier particles and the sediment [17A, 46A, 47A]. Our elution tests in the capillary system at a superficial velocity of 8.3×10⁻⁴ m/s indicate that almost all the particles elute through the capillary. Calculations of the sticking coefficient (Supporting Information) indicate values in the range of 0.03-0.08 with 97-99% elution. Phenrat and coworkers have conducted a comprehensive study of polyelectrolyte modified NZVI particles eluting through packed columns, and have postulated sticking coefficients in the range 10⁻² to 10⁻³ [47A]. The results suggest that polyelectrolyte modified NZVI transport is controlled by filtration theory as modeled by the T-E. We propose that since the carbon particles are covered by CMC, the same conclusions apply with the T-E equation governing the mobility. The efficient transport through the capillary also suggests negligible sticking to the sand grains.

### Particle Characteristics

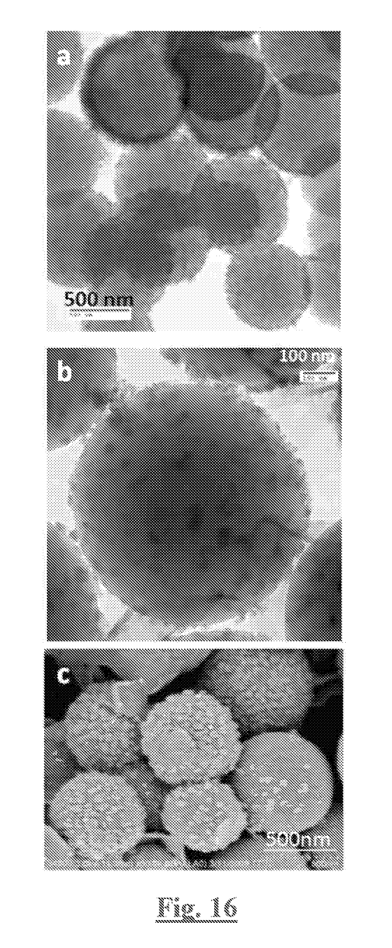

The morphology and microstructure of these multifunctional particulate systems were analyzed through transmission and scanning electron microscopy. As shown in FIGS. 11a and 11b, carbon particles prepared through the hydrothermal and pyrolysis process are monodisperse, uniform, and spherical with particle size around 500 nm to 5 μm (around 50 nm to 6 μm; preferably 200 nm to 6 μm; more preferably 200 nm to 1.5 μm; even more preferably 300-700 nm; most preferably 400-600 nm; e.g. 500 nm), consistent with the literature [29A]. FIGS. 16a and 16b illustrate the TEMs of the carbon particles wrapped with NZVI containing CMC, the (CMC+NZVI)/carbon system. The NZVI particles are visualized clearly due to the high electron density of iron. FIG. 16c illustrates the SEM of the composite particles showing a clear difference in morphology from the bare carbon. We do not however, consider the SEM an accurate representation of the system, since the drying of the system prior to imaging and the gold coating on the polymer+NZVI layer creates images that are somewhat artificial. Nevertheless, there is evidence of a significantly particle-flecked surface that is very distinct from that of pristine carbon microspheres.

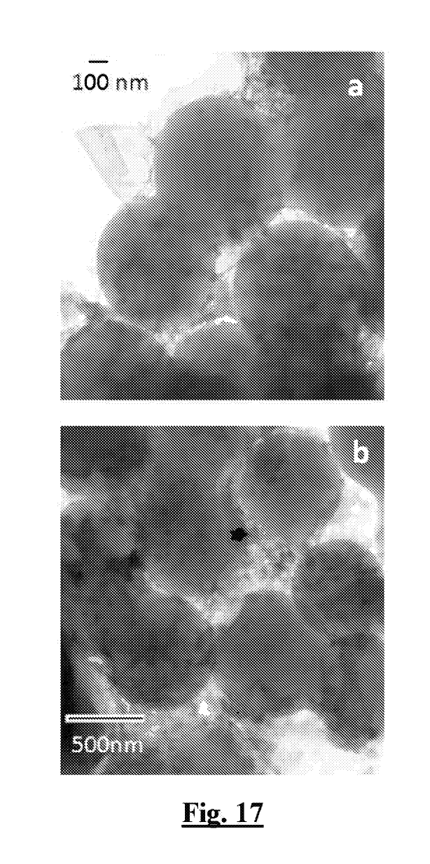

FIG. 17 provides TEM images of the CMC+NZVI+carbon system before and after transport through the capillary. The similarity between the two figures adds evidence to the hypothesis that the carbon microspheres are able to transport CMC loaded with NZVI through the sediment. An alternative technology that we are evaluating is the actual immobilization of NZVI on the carbon microspheres followed by system stabilization with CMC. We also note that the system of carbon microspheres can be easily extrapolated to other polyelectrolytes with attached NZVI, or to commercially available materials such as the modified reactive nanoscale iron particles manufactured by Toda Kogyo Corp. (M-RNIP). The commercial nanoscale iron particles we have, have lost their activity over time (this is natural, as the iron gets oxidized over time). However, we are able to stabilize them together with the carbon particles by using CMC. After further experimentation, we have been able to reactivate the commercial nanoscale iron particles with the use of sodium borohydride. Keeping iron in a zerovalent state furthers the remediation process.

To summarize, the present invention includes a multifunctional CMC stabilized NZVI+carbon microsphere based colloidal system for remediation of DNAPLs such as TCE. The system is able to sequester and break down TCE simultaneously, as well as move through the subsurface readily and partition to TCE phase easily. Considering that the preparation process is simple and made with inexpensive precursors and can be easily scaled up as a solution process, the system may hold promise in field testing. Such studies need to be done to evaluate the full potential of the system. The carbon based systems also have potential in reactive barrier applications.

The first technology using hydrothermal dehydration produces uniformly sized particles (monodisperse particles). Monodisperse particles are extremely useful for the lubrication application. However for the TCE dechlorination application where large quantities of material are required, this technology is believed to be not as efficient. The second technology using the aerosol-based process is much more efficient in making carbon particles with zerovalent iron on/in them. However, the particles are not monodisperse and there is a size distribution between 100 nm and 2000 nm typically. The particle size is within the range for optimal transport of the particles through groundwater saturated sediments. Hence the aerosol method is very useful for the TCE dechlorination application.

### Technology II:

### Experimental Methods

Materials.

Chemicals including sucrose (ACS reagent), ferric chloride hexahydrate (FeCl₃.6H₂O), sodium borohydride (NaBH₄, 99%) and trichloroethylene (TCE, 99%) were purchased from Sigma-Aldrich. Sulfuric acid (H₂SO₄, certified ACS Plus) was from Fisher Scientific. All chemicals were used as received without any further treatment. Deionized (DI) water generated with a Barnstead E-pure purifier (IA) to a resistance of approximately 18 MS2 was used in all experiments.

Sample Preparation.

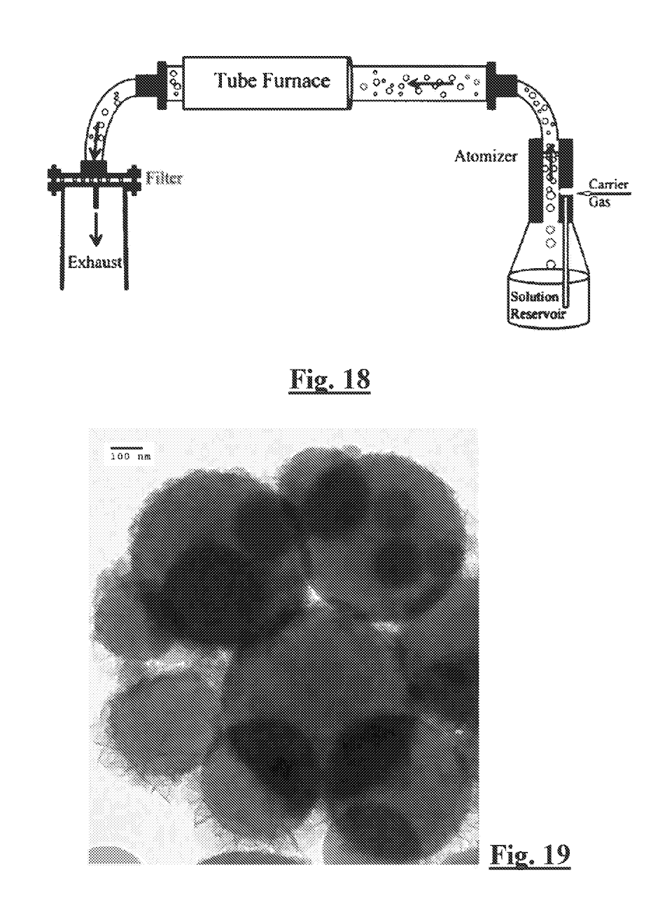

In a typical synthesis, 7.0 g of sucrose and 3.0 g of FeCl₃.6H₂O were dissolved in 35 mL of water. To this solution, 0.7 g of concentrated H₂SO₄ was added. The aerosol process was carried out in an apparatus depicted in FIG. 18. The precursor was first atomized to form aerosol droplets, which were then sent through a drying zone and heating zone where preliminary solvent evaporation and sugar carbonization occur. The temperature of the heating zone was held at 300° C. The resulting particles were collected by a filter maintained at 100° C. FIG. 18 is a representation of the solidification reaction in an aerosol droplet containing the entrapped iron species with solvent evaporation and sugar carbonization.

Ferric chloride in the as-synthesized particles was reduced to ZVI through liquid phase NaBH₄ reduction. In detail, 0.5 g of particles was put into a 15 mL centrifuge vial followed by drop-wise addition of a 10 mL NaBH₄ water solution (30 g/L). When hydrogen evolution ceased, the particles were centrifuged and washed by water several times before use.

Characterization.

Transmission electron microscopy (TEM, JEOL 2010, operated at 120 kV voltage) and field emission scanning electron microscopy (SEM, Hitachi S-4800, operated at 20 kV) were used to characterize the morphology of the particles. X-ray powder diffraction (XRD) was performed using Siemens D 500 diffractometer with Cu Kα radiation at 1.54 Å. The porosity of the particles was measured by the nitrogen sorption technique at 77K (Micromeritics, ASAP 2010). The samples were degassed at 200° C. prior to the measurement. Specific surface areas were determined using the Brunauer-Emmett-Teller (BET) equation.

Analytical.

TCE dechlorination effectiveness was tested in batch experiments. In detail, 0.5 g of the particles after reduction was dispersed in 20 mL water and placed in a 40 mL reaction vial capped with a Mininert valve. To this vial, 20 μL of a TCE stock solution (20 g/L TCE in methanol) was spiked into the solution containing the nanoparticles, which resulted in an initial TCE concentration of 20 ppm. The reaction was monitored through headspace analysis using a HP 6890 gas chromatography (GC) equipped with a J&W Scientific capillary column (30 m×0.32 mm) and flame ionization detector (FID). Samples were injected splitless at 220° C. The oven temperature was held at 75° C. for 2 min, ramped to 150° C. at a rate of 25° C./min and finally held at 150° C. for 10 min to ensure adequate peak separation between TCE, chlorinated and non-chlorinated reaction products.

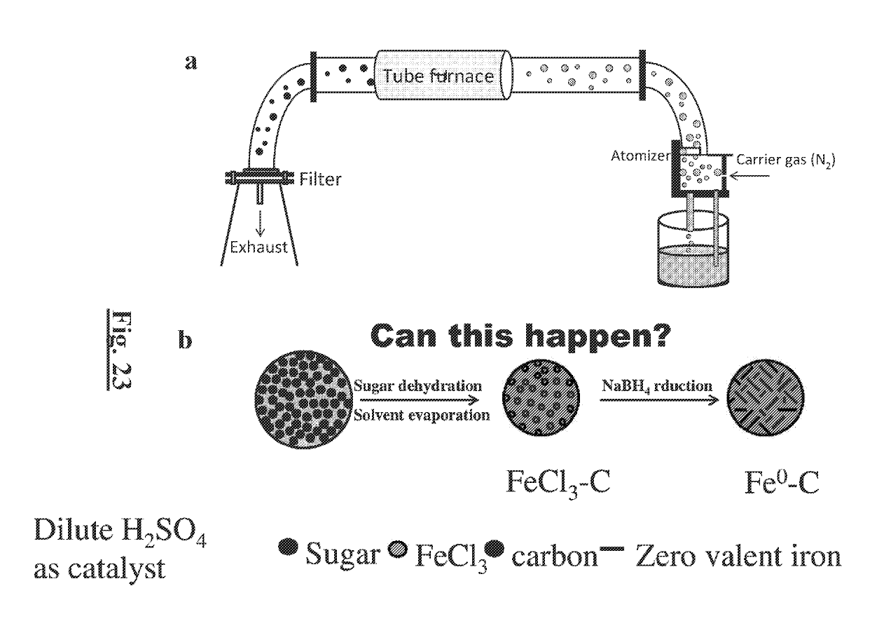

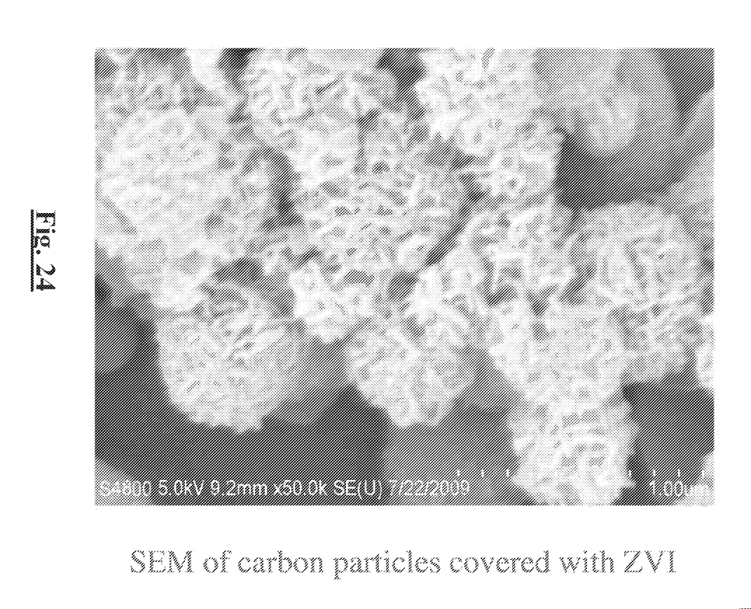

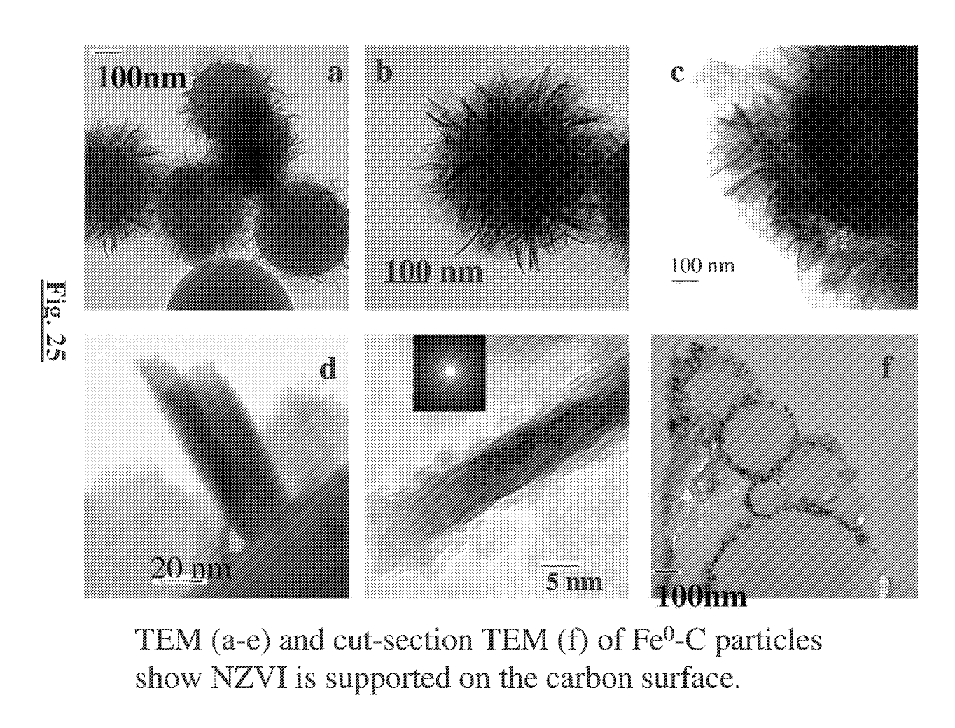

FIG. 23 shows how the aerosol process is done for Technology II. FIG. 24 is a scanning electron micrograph (SEM) of the carbon particles covered with the iron. Note that the iron is in the form of needles. FIG. 25 are transmission electron micrographs (TEM) of the particles at increasing resolution (magnifications) up to “e”. Note the needles again, and the fact that the increasing magnifications focus on looking at the needles. The needle shaped morphologies are rather unique. In “f”, we do a cut section TEM and it appears that the iron is only on the outside of the carbon particles. We also note that the carbon particles are not monodisperse.

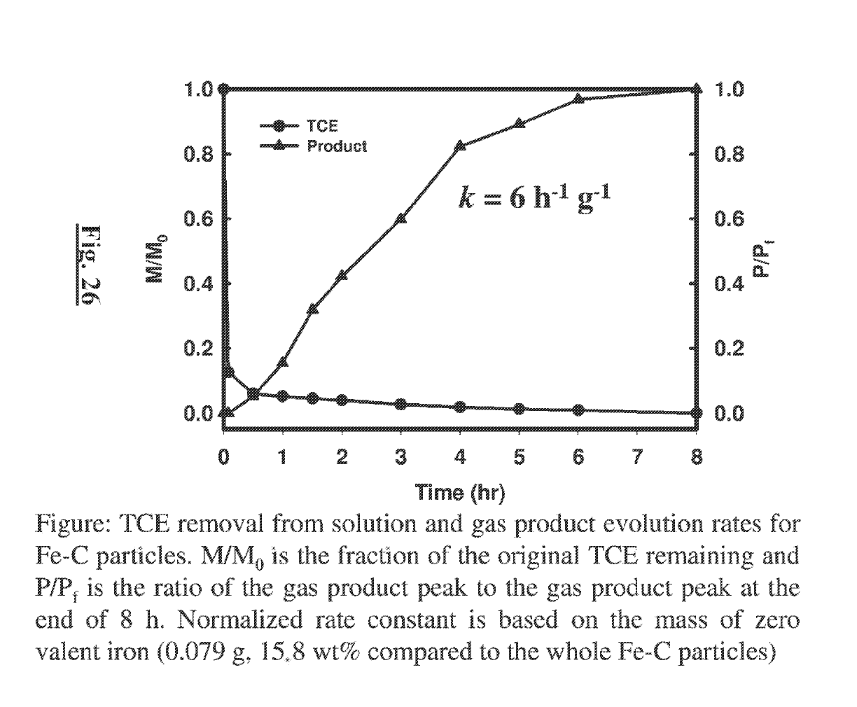

FIG. 26 shows the extremely rapid rate of destruction of TCE (gone in 8 hours) which is a distinctive feature of this technology. The sudden sharp decrease of TCE is because of the strong adsorption to carbon. FIG. 26 shows TCE removal from solution and gas product evolution rates for Fe—C particles. M/M₀ is the fraction of the original TCE remaining and P/P_{f} is the ratio of the gas product peak to the gas product peak at the end of 8 h. Normalized rate constant is based on the mass of zero valent iron (0.079 g, 15.8 wt % compared to the whole Fe—C particles).

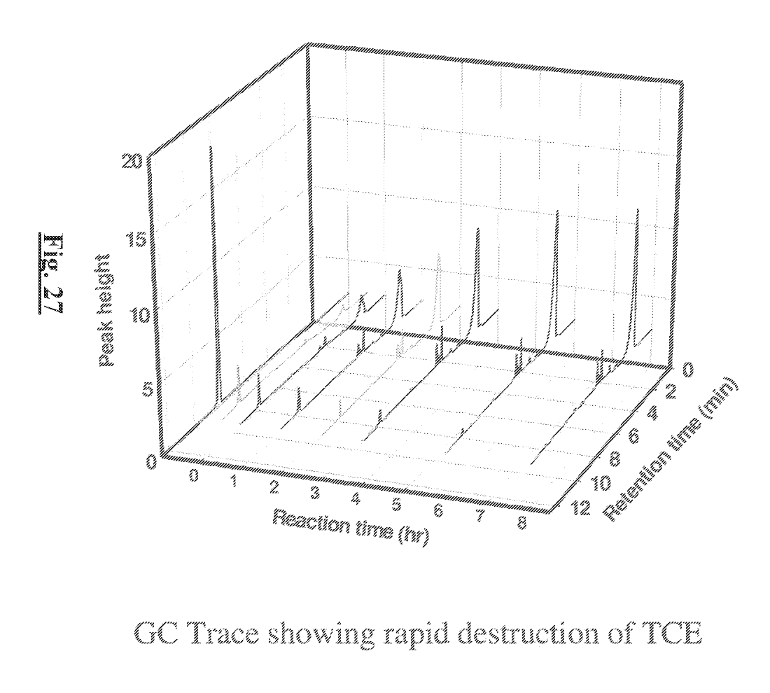

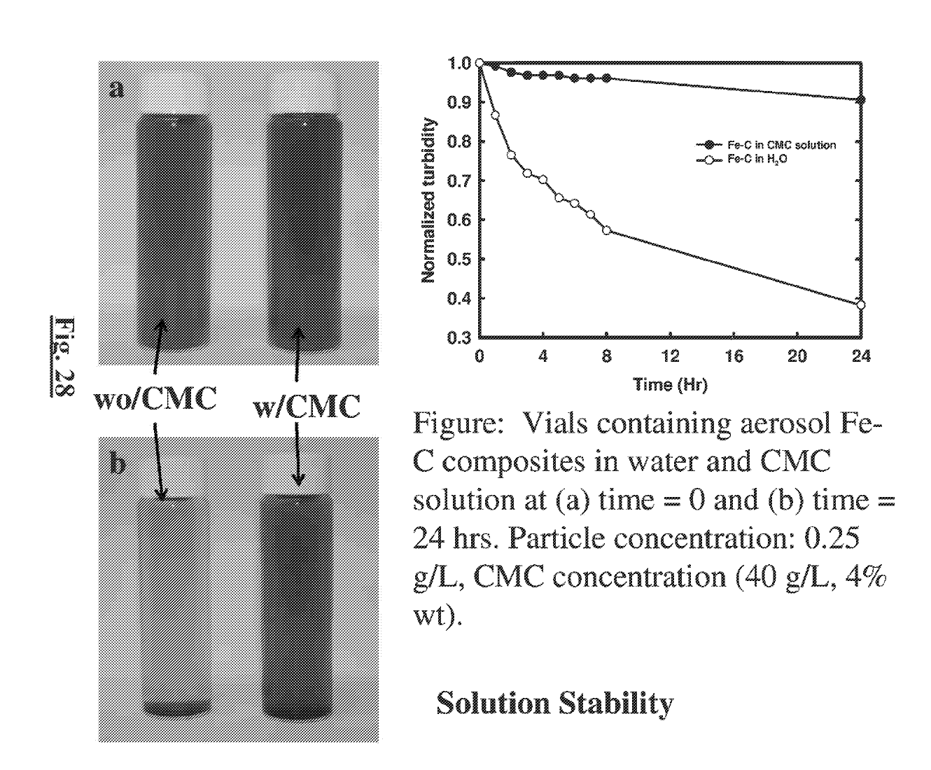

FIG. 27 is a gas chromatographic trace of the fast reaction. At time 0, before contact with the particles, we see a big TCE peak. As soon as the TCE is contacted with the particles, the TCE peak drops (due to adsorption) and the products begin to rise. At the end (8 hours) we only see the products and negligible TCE. In FIG. 28, we are showing here that the carbon (+iron) particles are stabilized by the addition of CMC. In this figure, vials are shown containing aerosol Fe—C composites in water and CMC solution at (a) time=0 and (b) time=24 hrs. The particle concentration is 0.25 g/L, CMC concentration is 40 g/L, 4% wt.

There is no need for palladium or other catalyst likely because the morphology of the iron particles are needle-shaped instead of circular-shaped, which makes it more highly reactive. However, it is possible to add the Pd or other catalyst to speed up the process more.

Technology II appears to be very promising due to: (a) high throughput rates through the aerosol process; (b) relatively high reactivity; (c) avoidance of Pd (or other catalyst due to the high reactivity).

Our continuing studies seek to understand the reasons for the relatively high reactivities, the morphological observations, and scaleup issues. We have chosen this system as the primary focus of studies on environmental impact. (Upstream—manufacturing issues, Downstream—environmental fate and transport).

### Aerosol-Based Method to Prepare Efficient Carbon Supported Zerovalent Iron Particles for Environmental Remediation of Chlorinated Hydrocarbons

The present invention also includes an aerosol-based method to prepare efficient carbon supported zerovalent iron particles for environmental remediation of chlorinated hydrocarbons.

The following steps to manufacture are proposed.

1. The use of an aerosol reactor or conventional spray drier technology. The aerosol reactor is where liquid is sucked into chamber where it is made into droplets. The spray drier uses air to push the substance through a nozzle and into a chamber to dry by Nitrogen or Ar. The feed stream can be a common sugar (sucrose) or any variety of saccharides or poly-saccharides (glucose, cellulose, cyclodextrins) with dilute sulfuric acid to enable the dehydration. Additionally an iron precursor (e.g. FeCl₃) is preferably added to the feed. The feed stream is passed through a nozzle for aerosolization and then through a heated zone in a furnace, kept at temperatures between 100 and 400° C. During passage in the heated zone, the droplets evaporate and the sugars become dehydrated.

FIG. 18 illustrates the aerosol reactor.

At the exit of the heating zone the particles are collected on a filter. The exhaust is vented out. The process is semicontinuous and can be scaled up to produce large quantities of particles.

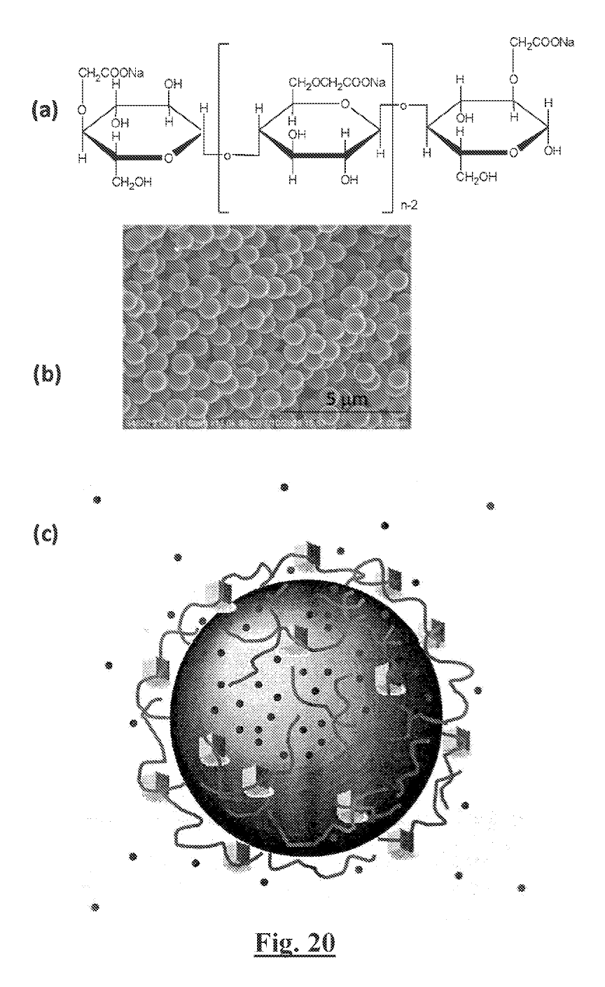

2. Once the particles are collected they are dispersed in aqueous solution to which we add sodium borohydride to reduce the iron oxides and iron hydroxides to zerovalent iron. The resulting particles are shown in FIG. 19. Hydrazine can also be used instead of the sodium borohydride solution (but its toxic), as well as a variety of polyphenols. The use of polyphenols has been proposed by others. We have not yet tried polyphenols, but they may work.

These particles are highly reactive to TCE and break down TCE extremely easily. They are also strong adsorbents for chlorinated hydrocarbons.

3. In order to stabilize these particles, we add a polyelectrolyte such as carboxymethyl cellulose (CMC). The polyelectrolyte is added to the iron and carbon right before injecting the particles into the ground. The polyelectrolyte can also be combined with the iron first, and then added to the carbon. Starch, Dextran, poly lactate, poly ascorbate, and modified chitosan are examples of biodegradable polyelectrolytes that can also be used instead of CMC. Gelatin and xantham gum may also work. Synthetic polymers include poly(acrylic acid) and poly (styrene sulfonate) but these are not biodegradable. The particles then become extremely stable in water and migrate with groundwater to the sites of contamination. They then partition to bulk TCE phases.

Precursor sugars for the aerosol method:

1. Monosaccharides such as sucrose, glucose, fructose, cyclodestrin

2. Polysaccharides such as cellulose, dextran, carboxymethyl cellulose, starch.

Differences from the Earlier Disclosed Embodiment:

1. This is a one step procedure to prepare iron and carbon together. The earlier disclosed embodiment involved first preparing the carbon separately and then adding it to CMC+iron.

2. The current procedure is more easily scalable than the earlier one since it is done in a semicontinuous system (the aerosol reactor or a spray drier). The earlier procedure is a batch process involving solution chemistry. However, the earlier process does not use dilute sulfuric acid and because it is solution based, could be a bit safer. However with the aerosol procedure, it is very easy to scale up to make the large quantities necessary for field application.

3. Reaction rates for TCE destruction appear to be extremely fast with the new procedure (i.e., aerosol procedure) in comparison with the earlier disclosed procedure (i.e., hydrothermal process). The reaction rates between the two procedures are substantially different without palladium. The reaction rates between the two procedures are more similar with the addition of palladium.

4. The aerosol process entraps the iron onto the carbon more securely. In the non-aerosol method, the iron disperses from the carbon easily.

The hypothesis is that the aerosol process creates an enhanced adhesive contact between the iron particles and the carbons because the process inherently forces the iron nanoparticles to attach to the carbon as the droplet evaporates. Alternate techniques in the literature (e.g., Schrick, B.; Hydutsky, B. W.; Blough, J. L.; Mallouk, T. E., Delivery Vehicles for Zerovalent Metal Nanoparticles in Soil and Groundwater. Chem. Mater. 2004, 16, (11), 2187-2193) are less effective in placing the nanoparticles on carbon.

### Multifunctional Iron-Carbon Nanocomposites Through an Aerosol-Based Process for the in Situ Remediation of Chlorinated Hydrocarbons:

In recent years, extensive efforts have been carried out to develop and synthesize nanomaterials with unique reactivity and functional characteristics for environmental applications [1C-9C]. For example, the use of nanoscale zero-valent iron (NZVI) particles represents a promising approach to the remediation of chlorinated organic-contaminated groundwater such as trichloroethylene (TCE) [10C-12C]. Compared to conventional micro-scale granular iron powders, the advantages of using NZVI particles include the potentially high reactivity as a consequence of high surface areas, and the fact that they can be colloidally stabilized, suspended as a slurry and injected into the subsurface [13C-16C]. However, the intrinsic ferromagnetism of NZVI particles leads to aggregation and there continues to be difficulties in developing efficient in situ technologies [17C-19C].

Several criteria need to be met in the design of effective systems for in situ degradation of chlorinated compounds. Such systems must be able to move through the subsurface with high mobility, show affinity towards hydrophobic TCE, and break down the contaminant efficiently. The mobility of colloids in the subsurface is determined by competitive mechanisms of Brownian motion, interception by soil and sediment grains and sedimentation effects [20C]. The Tufenkji-Elimelech model, which considers the effect of hydrodynamic forces and Van der Waals interactions between colloidal particles and sediment grains predicts that particles in the size range 0.1 to 1.0 microns are likely to be the most mobile at typical groundwater flow conditions [21C-23C]. In addition, considering that hydrophobic organic contaminants are retained by soil grains via capillary forces and adsorption [22C], it would be advantageous if these particles also reduce the concentration of dissolved TCE through a combination of sequestration by adsorption followed by degradation of TCE. Commonly, the mobility of NZVI particles can be enhanced by adsorption of hydrophilic or amphiphilic organic species such as surfactants, vegetable oils, starch, or polyelectrolytes such as carboxymethyl cellulose (CMC) and poly (acrylic acid) (PAA), or triblock copolymers on the NZVI particle surface [18C, 22C, 24C-28C]. These adsorbed organics inhibit NZVI aggregation and enhance solution stability through steric hindrance and/or electrostatic repulsion [29C, 30C]. Alternatively, NZVI immobilized onto support materials such as activated carbon granules (1-3 mm) are an effective way to inhibit aggregation of nanoscale zero-valent iron particles [31C]. Composites with carbon introduce a strong adsorptive aspect into remediation technology as the carbon adsorbs chlorinated compounds, and these materials have been used in the development of adsorptive-reactive barriers [32C, 33C]. In a pioneering paper on the use of carbon, Shrick and coworkers have shown that carbon black is a useful additive to zerovalent iron to prevent aggregation and facilitate reaction and transport. [22C]

In recent work from our laboratory, we have shown the preparation of monodisperse carbon particles obtained from the hydrothermal dehydration and pyrolysis of sugar, as supports for zerovalent iron [34C]. While the process is feasible and leads to an effective system, the preparation of the Fe-carbon composite is a multiple step process. We describe here, the facile preparation of a multifunctional particulate system containing zero-valent iron that has the requisite characteristics of reaction, adsorption and transport to effectively address the degradation of chlorinated compounds. In addition, the particulate system is obtained from inexpensive precursors and through a semi-continuous method which allows large scale synthesis necessary for eventual in situ application. The particulates contain NZVI supported on carbon microspheres and are synthesized through an aerosol route using inexpensive sugars as precursors. Prior studies have demonstrated that the aerosol-based technology is a simple approach to prepare silica particles in the submicron (typically 100-800 nm) size range [23C, 35C-37C]. In the present invention, we expand the aerosol-based technology to produce carbon-based functional nanocomposites of zero-valent iron supported on carbon spheres. The postulates of the work are the following: (i) immobilization of NZVI onto carbon spheres may make the ZVI less prone to aggregation, while maintaining reactivity; (ii) carbon produced by an aerosol-based process serves as a strong adsorbent for TCE increasing local concentrations at the ZVI reaction sites thereby enhancing the driving force of reaction; (iii) the aerosol-based process is an efficient method to synthesize such multifunctional adsorptive-reactive materials in the optimal size range for transport through sediments. Additionally, the semi-continuous nature of the aerosol process indicates the feasibility of scale up. To the best of our knowledge, this is the first report of a one-step method of preparing multifunctional materials for use in the reductive dechlorination of dense non-aqueous phase chlorinated compounds.

### Experimental Section

### Materials.

Chemicals including sucrose (ACS reagent), ferric chloride hexahydrate (FeCl₃.6H₂O), sodium borohydride (NaBH₄, 99%) and trichloroethylene (TCE, 99%) were purchased from Sigma-Aldrich. Sulfuric acid (H₂SO₄, certified ACS Plus) was from Fisher Scientific. All chemicals were used as received without further treatment. Deionized (DI) water, generated with a Barnstead E-pure purifier (IA) to a resistance of approximately 18 MQ, was used in all experiments.

### Sample Preparation.

The aerosol-based technique was employed to prepare iron-carbon composites. In a typical synthesis, 7.0 g of sucrose and 3.0 g of FeCl₃.6H₂O were dissolved in 35 mL of water. To this solution, 0.7 g of concentrated H₂SO₄ (2% w/v) was added. The use of H₂SO₄ as a catalyst for dehydration of sugar is only required at low temperatures of carbonization. The resulting solution was aged for 30 min under stirring to mix the solution completely. In the aerosol-based process, the precursor was first atomized to form aerosol droplets, which were then carried by an inert gas (N₂) through a heating zone where solvent evaporation and carbonization occurs. The flow rate of the carrier gas was 2.5 L/min and the heating was done in a 100 cm tube with a furnace length of 38 cm leading to a superficial velocity of 2.7 cm/s. The temperature of the heating zone was held at 350° C. The resulting Fe salt/carbon particles were collected over a filter maintained at 100° C.

Ferric iron salt in the as-synthesized Fe salt/carbon particles was reduced to ZVI through liquid phase NaBH₄ reduction as the previously reported [10C, 37C]. Specifically, 0.5 g of particles collected from filter paper was put into a vial followed by drop-wise addition of a 10 mL of 0.8 M NaBH₄ water solution. After cessation of visible hydrogen evolution, the particles were centrifuged and washed by water thoroughly before use. The control sample is that of aerosol-based bare carbon particles without the use of the iron precursor.

### Characterization and Analysis.

Transmission electron microscopy (TEM, JEOL 2010, operated at 200 kV voltage) and field emission scanning electron microscopy (SEM, Hitachi S-4800, operated at 20 kV) were used to characterize the morphology of the particles. X-ray powder diffraction (XRD) was performed using a Siemens D 500 diffractometer with Cu Kα radiation at 1.54 Å. X-ray photoelectron spectroscopy (XPS) was conducted with a Scienta ESCA-300 high-solution X-ray photoelectron spectrometer (HR-XPS). A Kα X-ray beam at 3.8 kW was generated from an A1 rotating anode. Optical microscopy (Olympus IX71, Japan) were used to characterize the transport properties of the composites through packed capillaries. In analysis, TCE dechlorination effectiveness was tested in batch experiments. In detail, 0.5 g of the aerosol-based Fe/C composites were dispersed in 20 mL water and placed in a 40 mL reaction vial capped with a Mininert valve. To this vial, 20 μL of a TCE stock solution (20 g/L TCE in methanol) were spiked, resulting in an initial TCE concentration of 20 ppm. The reaction was monitored through headspace analysis using the procedures described in our earlier work [38C, 39C].

### Results and Discussions

### Synthesis and Characterization.

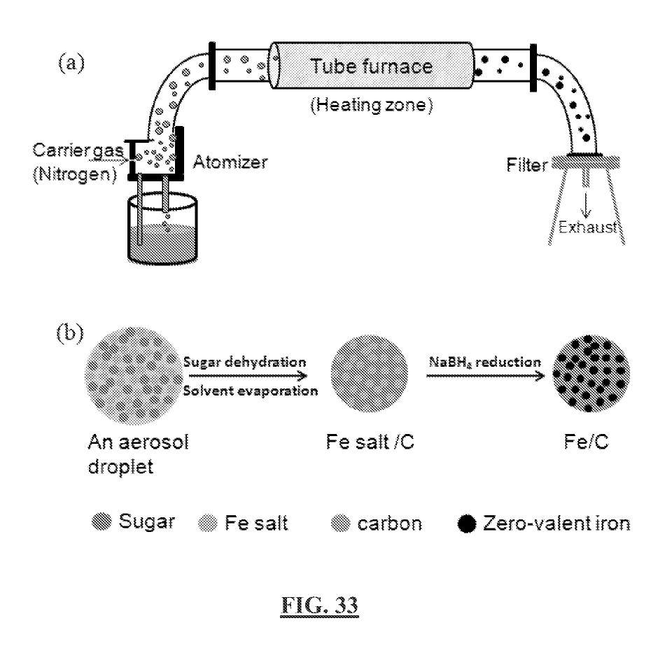

We adopt here the nomenclature of Fe/C to depict NZVI particles supported on the carbon material prepared via an aerosol-based process. FIG. 33a the schematic of the aerosol reactor, consisting of an atomizer, a heating zone and a filter. Starting with a homogenous aqueous solution containing sucrose, iron chloride and sulfuric acid, a commercial atomizer (Model 3076, TSI, Inc., St Paul, Minn.) atomizes the solution into droplets that undergo a heating and drying step, generating submicron particles that are collected on a filter. FIG. 33b is a representation of the formation route of Fe/C composites. When the aerosol droplets pass through the heating zone, solvent evaporation and dehydration/carbonization of sucrose occur. The role of sulfuric acid (when used) is to accelerate the process of carbonization especially at lower furnace temperatures. In addition, precipitation of solidified iron salt is concomitant with the dehydration of sucrose, generating a black powder of Fe salt/C composites. To obtain Fe/C composites, the collected powder is treated with sodium borohydride solution in excess to reduce ferric ion to zero-valent iron. The final weight percentage of zero-valent iron in the Fe/C composites was approximately 15%. This content was determined by weighing the residual solid (Fe₂O₃) of a known mass of Fe/C composites after calcination in air for 4 hours at 500° C. to burn off the carbon.

It is note-worthy that operating conditions for synthesis of the aerosol-based Fe/C composites are adjustable. For instance, Fe/C composites can be obtained at temperatures as low as 350° C. with dilute sulfuric acid added to the precursor solution to catalyze carbonization, or at higher temperatures without any sulfuric acid. In all cases, we have found Fe/C composites with the requisite characteristics; for brevity we report the characteristics of particles synthesized at 350° C. with dilute sulfuric acid addition.

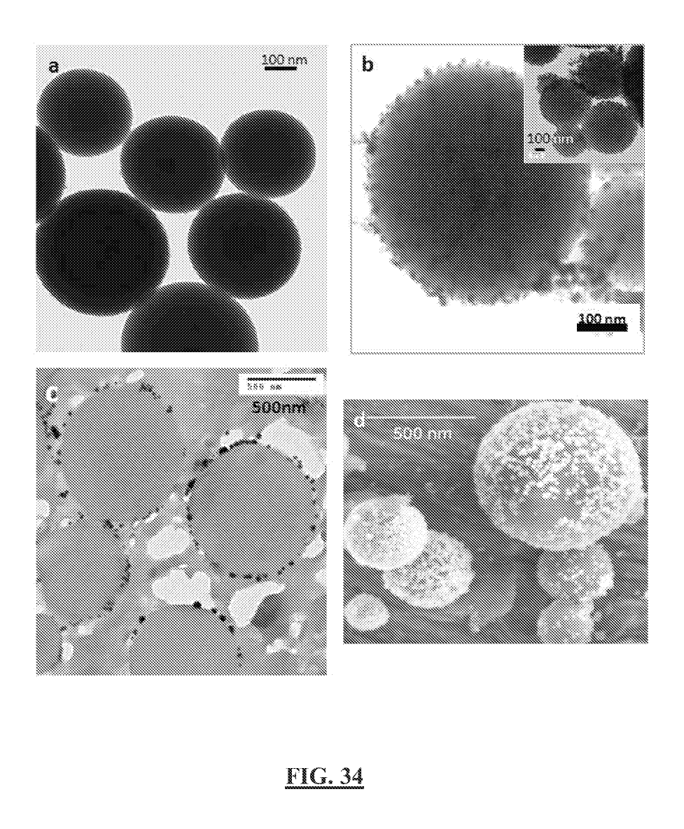

The microstructure and morphology of the multifunctional nanostructured particles were analyzed through transmission and scanning electron microscopy. FIG. 34a shows the TEM image of aerosol-based bare carbon particles as the control. The particles are well-defined microspheres. For Fe/C composites as shown in FIG. 34b, the presence of NZVI with higher electron contrast on carbon supports indicates distribution of nanoiron throughout the surface of carbon, with the average size around 15 nm; the lack of aggregation to large clusters is noted, in contrast to other methods of synthesizing NZVI for dechlorination [40C]. The fact that NZVI particles are attached on the surface of carbon was further confirmed by the cut-section TEM image as shown in FIG. 34c. To prepare the cut-section TEM, the samples were embedded in an epoxy resin, dried overnight, and microtomed into thin slices (approximately 70 nm) with a diamond knife. A thin slice of the microtomed sample was transferred to a copper grid and the sequent procedures completely followed the normal TEM process. Clearly, the cut-section TEM image shows a strong contrast between the dark edge and pale core, implying that zero-valent iron nanoparticles are attached to the surface rather than located in the interior. In agreement with the SEM image (FIG. 34d), it can be seen that all nanostructured Fe/C particles are spherical and discrete zero-valent iron nanoparticles are decorated on the surface of carbon spheres. XRD and XPS data (shown in the supporting information) further indicate the presence of zero-valent iron.

### Adsorption and Reactivity Studies.

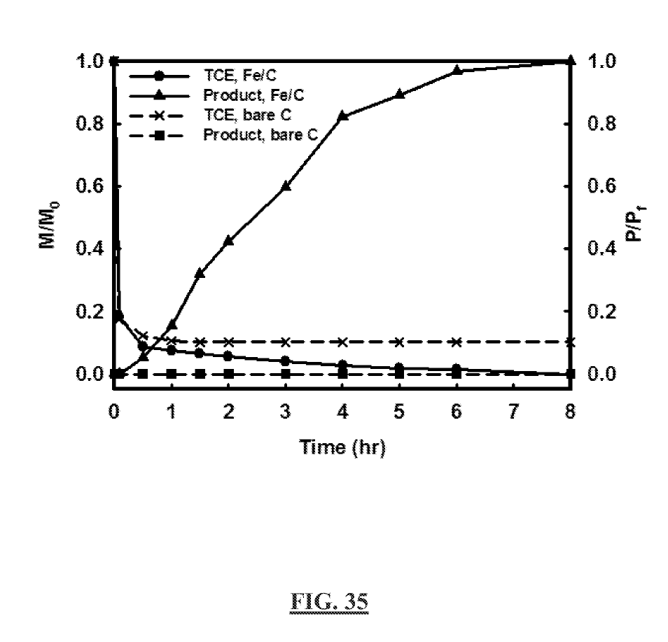

TCE removal from solution and gas product evolution rates are shown in FIG. 35, where the performance of Fe/C and bare carbon when contacted with dissolved TCE are compared. We note an immediate sharp decrease of the dissolved TCE concentration to 18% of its original value followed by a much slower decrease. The initial sharp decrease is due to TCE partitioning from solution to the carbon through strong adsorption. This is an important aspect to the design of these materials as the phenomenon leads to enhanced reactant concentrations in the vicinity of the reactive NZVI sites. The rate at which gas products evolve is indicative of the observed reaction kinetics of TCE. To prove the concept that the carbon is a strong adsorbent for TCE, we exposed bare aerosol carbon particles to TCE-containing solutions. As expected, there is an immediate and sharp reduction of solution concentration with an average adsorption of 0.66 mg of TCE/(g of aerosol carbon) (or 82.5% of the total TCE) at the TCE concentration used in this study, but no further decrease in concentration due to reaction.

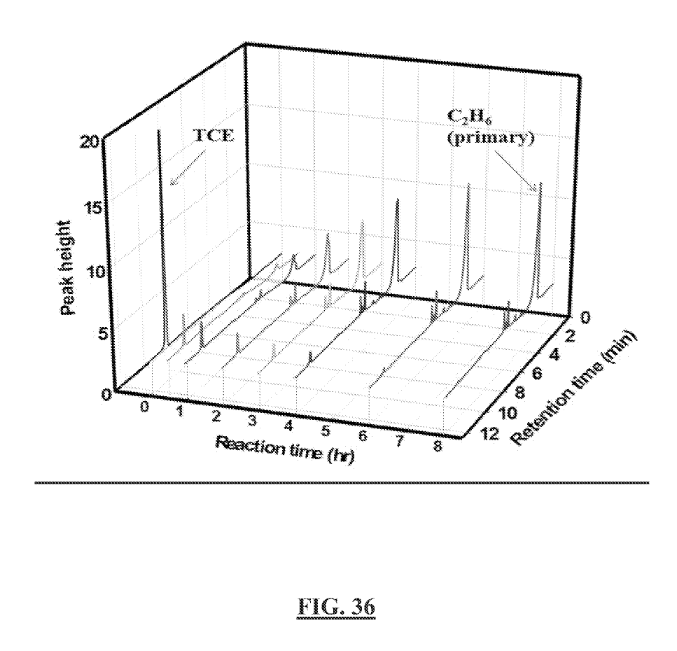

Since adsorption is rapid, reaction is the rate controlling step and it is possible to calculate a pseudo-first order rate constant by following the evolution of the lumped gas phase products [38C, 39C]. For Fe/C composites, the apparent reaction rate constant, k_{obs} is approximately 0.47 h⁻¹ with the mass-normalized reaction constant, kₘ is 0.12 L hr⁻¹ g⁻¹ based on the mass of zero-valent iron. In contrast, commonly reported rate constants for NZVI for the remediation of TCE are 0.013 h⁻¹ and 0.0026 L hr⁻¹ g⁻¹, respectively [41C]. The ˜45-fold difference in kₘ suggests that the application of Fe/C not only provides a strong sequestration mechanism, but also greatly enhances the reactivity. The enhanced reactivity could be due to (a) the high surface area of non-aggregated NZVI (b) the increased local concentrations of TCE due to adsorption. FIG. 36 illustrates the time evolution of gas phase products. The chromatogram is illustrative in that it shows the sharp decrease of solution TCE level as soon as the reactive particles are added, a consequence of TCE adsorption. It is interesting to note that toxic intermediates such as dichloroethane (C₂H₂Cl₂) and vinyl chloride (C₂H₃Cl) are not observed, and the entire product range is based on the light gases. Again, this is due to the strong adsorptive characteristics of the carbon to sequester intermediates and we reemphasize the value of incorporating ZVI onto carbon particles. Any chlorinated intermediates remain adsorbed on the carbons until they are reacted away to the light gases, primarily ethane and ethylene, but including a small amount of butane and butene.

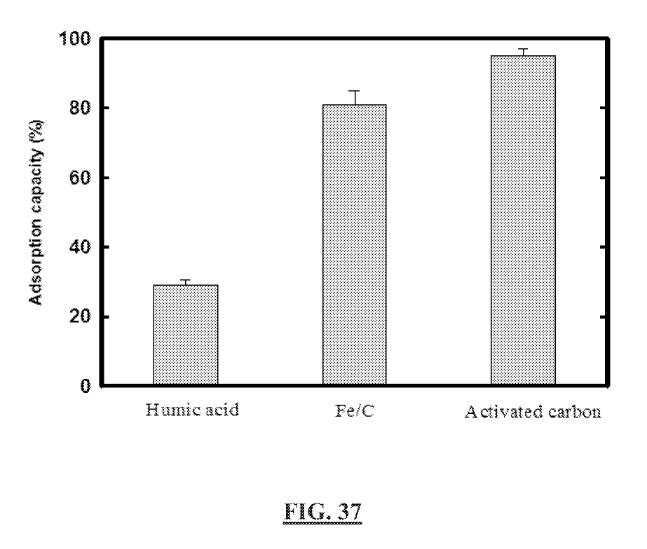

FIG. 37 compares adsorptive capacities of the aerosol-based Fe/C composites with humic acid (the major natural organic matter of soil) and commercial activated carbon. In all experiments, 20 mL of a 20 ppm TCE solution and 0.2 g of particles were used. The adsorption of TCE on Fe/C (˜85%) is higher than that of humic acid (˜30%) and comparable to that on commercially available granular and irregularly defined activated carbons (˜95%). The implication of the strong adsorption on the aerosol-based carbon is the ability to establish a driving force for chlorinated compounds to desorb from natural organic matter and partition to the carbon containing NZVI which leads to destruction of the TCE. This leads to highly effective remediation of contaminated sediments. We have calculated the partition coefficient for TCE adsorption on the aerosol-based Fe/C particles using the definition of Phenrat and coworkers [42C] and fully discussed in the supporting information. To summarize the measured partition coefficient for TCE adsorption on humic acid is 85, while Kₚ for the adsorption of TCE on aerosol-based Fe/C composites is 1560, an 18.3 fold increase in adsorption capacity. The implication is that even in systems containing TCE adsorbed to natural organic materials, there will be a driving force to transfer TCE to the Fe/C composites where they will be reacted away.

### Stability and Transport Characteristics.

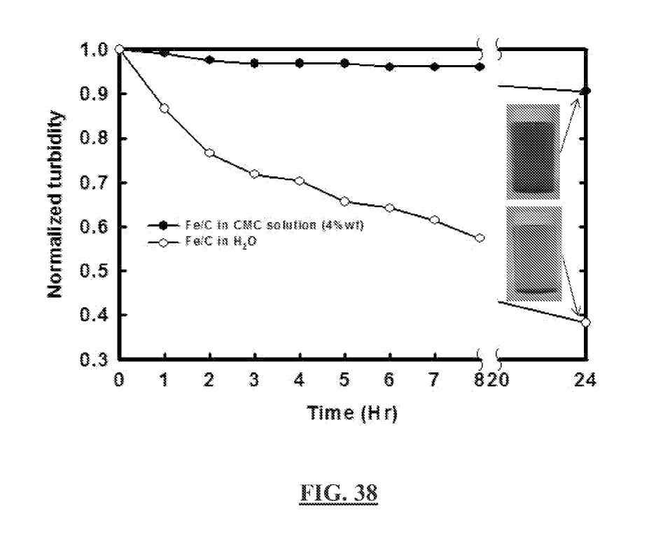

FIG. 38 demonstrates that colloidal stability of Fe/C particles can be significantly enhanced by the addition of polyelectrolytes such as carboxymethyl cellulose (CMC) a well-studied additive for colloidal stabilization through both steric and electrostatic repulsion effects [43C]. In the experiment, the initial concentration of Fe/C particles was maintained at 250 mg/L (0.01 g in 40 mL solution), and the content of CMC was 4% by weight. Sedimentation curves of suspensions were obtained by monitoring the turbidity of suspensions with a nephelometric turbidimeter (DRT100B, HF Scientific, Inc., Fort Myers, Fla.). The role of CMC in maintaining colloidal stability is clearly observed with over 90% of the particles remaining suspended after 24 hours. Increased amounts of CMC enhance stability further (data not shown here). We contrast the findings with results in the literature that indicate that bare NZVI particles rapidly aggregate and precipitate from solution in less than an hour, indicating the necessity to functionalize the NZVI or add colloidal stabilizers [17C, 44C, 45C]. In the technology described here, aggregation of NZVI is avoided by immobilization on carbon, and colloidal stability is brought about through the addition of an inexpensive polyelectrolyte.

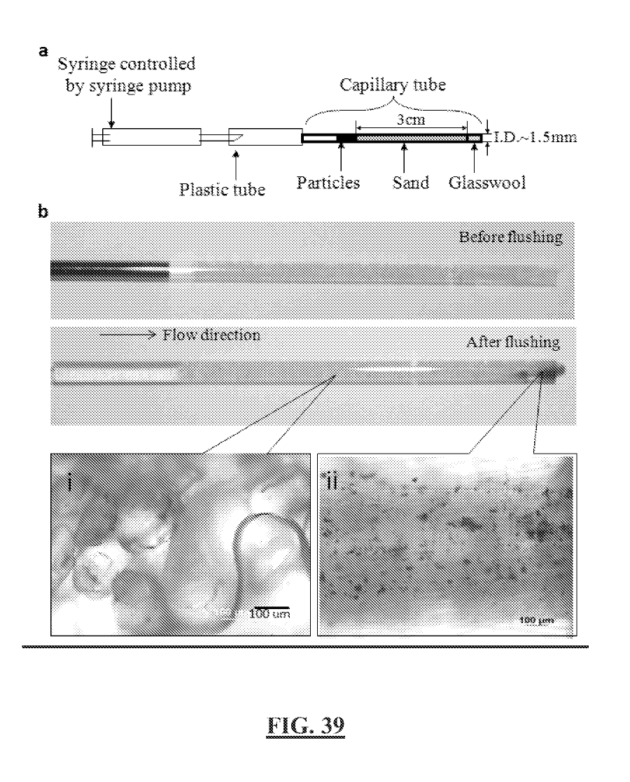

Transport characteristics of these multifunctional materials are examined through capillary transport experiments. The capillary experiment is an effective and intuitive method to study particle transport through porous media, and has been reported in our previous work [23C]. As shown in FIG. 39a, glass melting-point tubes with both ends open (1.5-1.8 mm i.d.×100 mm length, Corning, N.Y.) were used as capillaries. The capillary tubes were packed with wet Ottawa sand over a 3 cm length and were placed horizontally to simulate groundwater flow. A continuous water flow at a flow rate of 0.1 mL/min (Darcy velocity: 5 cm/min) was provided by a syringe pump. The exit point of the capillary was capped with a small glass wool plug. After 30 μL of CMC-stabilized Fe/C suspension was injected into the inlet of the capillary, water flushing was initiated and an inverted optical microscope was used to observe the pore-scale transport of the particles. FIG. 39b illustrates photographs of the capillaries depicting the capillary containing Fe/C colloids before and after the water flush. The images indicate that carbon supported NZVI particles readily transport through the packed capillaries and become captured in the glass wool. In contrast, bare NZVI particles agglomerate and do not transport through the capillary [23C]. With over 97% of the particles being eluted through the capillary, the sticking coefficient denoting the attachment probability of particles to the sediment is calculated at 0.09. Details of the sticking coefficient calculations are included in the Supporting Information.

In conclusion, nanoscale zero-valent iron particles have been supported on carbon particles using an aerosol-based process and subsequent reduction. These composites are specifically designed for use in the in situ breakdown of chlorinated hydrocarbons such as trichloroethylene (TCE). The following are beneficial characteristics of these systems: (1) the presence of nanoscale zero-valent iron in the composites ensures efficient TCE remediation (2) the aerosol-based carbon strongly adsorb TCE removing dissolved TCE rapidly and facilitate reaction by increasing TCE concentrations in the vicinity of the iron (3) the strongly adsorptive carbon prevents release of any toxic chlorinated intermediate (4) The particle size distribution is optimal for effective transport through soil (5) the composite particles are environmentally benign. Finally, the aerosol process is conducive to scale up as it is a virtually continuous process limited only by the batch requirements of particle collection on a filter.

The aerosol route to Fe/C composites is the most efficient technology we have developed. The following are variations that can be made in the process of producing the carbon microspheres:

### The Role of Sulfuric Acid

Sulfuric acid aids in carbonization of sugar to form carbon microspheres. At the lower temperatures of 300° C. and below, sulfuric acid is necessary to obtain the carbon microspheres. At temperatures of 500° C. and above, it is not necessary to use sulfuric acid. From an environmental and manufacturing perspective it is better to avoid the use of sulfuric acid.

### The Role of Temperature

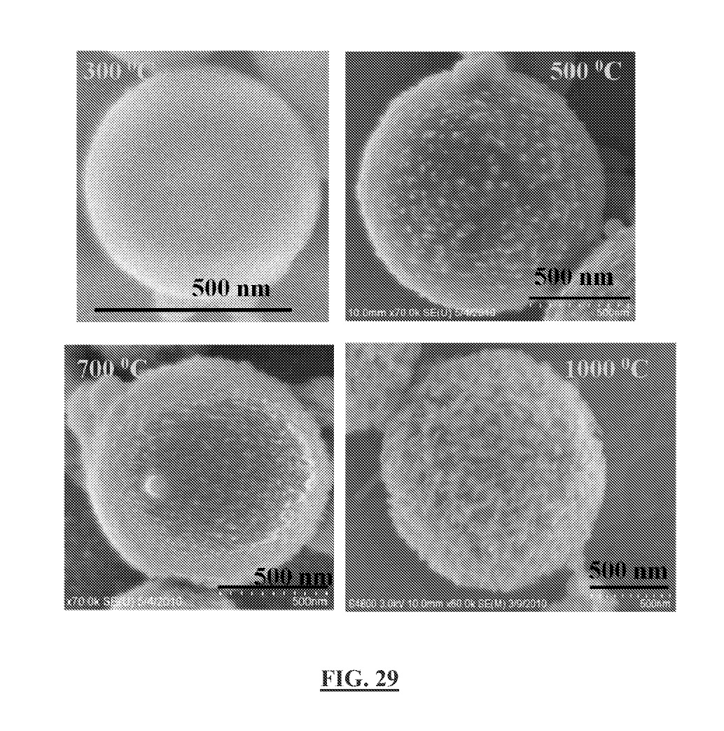

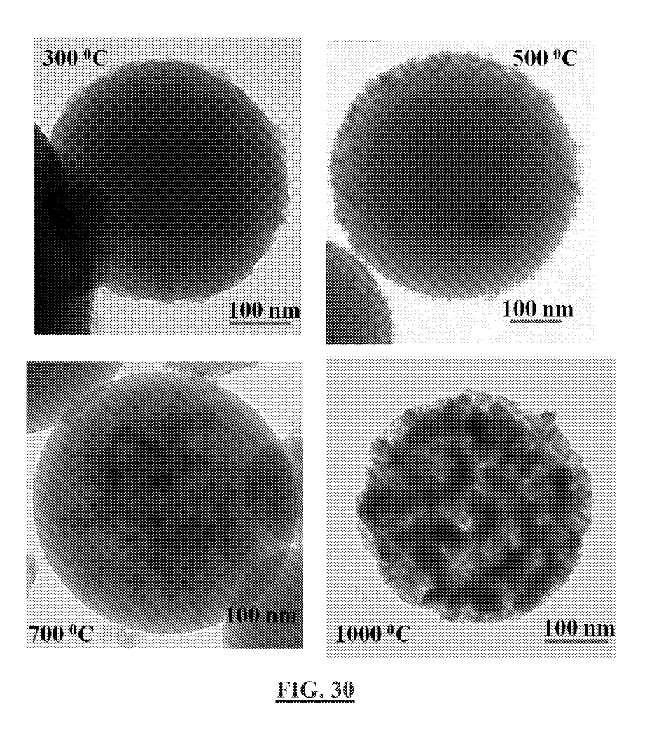

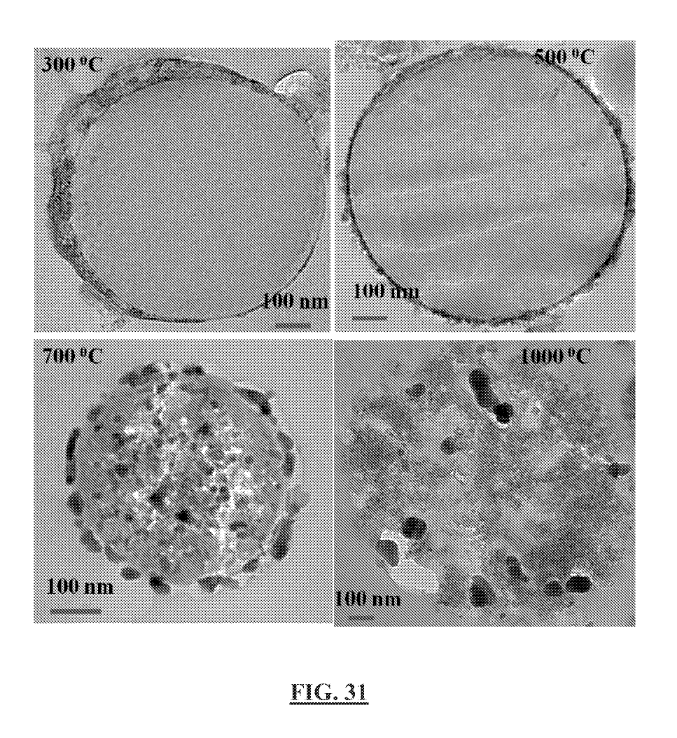

When the aerosolization is conducted at higher temperatures, one generates porous carbons. Furthermore, the iron is localized within the particles rather than on the surface. In other words, it is possible to control the placement of iron on the surface of the particle or in the interior using temperature. FIGS. 29-31 show how increased temperature affects the morphology of the particles. The scanning electron micrographs (SEM) (FIG. 29) show the surface morphology of the particles, the transmission electron micrograph (TEM) (FIG. 30) show the particles becoming progressively more porous as the synthesis temperature is increased and the location of the iron within the particles. The cut section TEM (FIG. 31) is the most illustrative where the iron nanoparticles (dark dots) are clearly located in the interior of the cut section and not on the surface.

### The Role of Sodium Borohydride

Sodium borohydride is a reductant. It takes the iron salts and reduces them to zerovalent iron. There are alternative methods of reduction (explained below). The question is whether sodium borohydride adds to the cost to the extent that the material becomes too expensive to use in large scale. However, field tests have been done with zerovalent iron formulations obtained through sodium borohydride reduction. There are several papers describing the use of sodium borohydrate and this is a well known reduction technique.

### The Role of Palladium

Palladium enhances the reaction rate in all cases. Every formulation containing zerovalent iron including nonreactive and minimally reactive formulations will have reaction rate enhancements through the use of Palladium. Typically 0.05-0.1 wt % Pd is added. Nickel has the same function but is not as effective. Typically up to 5 wt % Ni needs to be added to get the same rate enhancement as 0.1 wt % Pd. Again, this is not an idea developed in our laboratories but has been widely published in the literature.

Alternate Methods of Reduction include: FeCl₃ on carbon with aerosol method; FeSO₄ on carbon through aerosol method; Commercial nano Fe₂O₃ directly reduced; Fe Cl₃ on CMC; Fe Cl₃ directly reduced; FeSO₄ on hydrophilic carbon; FeOOH or Fe₂O₃; Fe(NO₃)₃ on activated carbon; Fe(NO₃)₃ on carbon black Fe(NO₃)₃ on Activated carbon.
1. Various precursor iron salts can be used, ferric chloride (FeCl₃), ferrous sulfate (FeSO₄), ferric nitrate Fe(NO₃)₃, ferric citrate, etc.
2. Various carbon sources can be used, such as
Monosaccharides such as sucrose, glucose, fructose, cyclodestrin
Polysaccharides such as cellulose, dextran, carboxymethyl cellulose, starch.
3. Addition of Pd always leads to greater activity.
4. Various methods of reduction (described next) can be used.

### Methods of Reduction:

1. The standard method of reduction is the use of sodium borohydride.
2. Alternate method of reduction is the carbothermal method.

### The Carbothermal for in Situ Remediation of Chlorinated Hydrocarbons:

We describe here, the facile preparation of a multifunctional particulate system containing zero-valent iron, which has the requisite characteristics of reaction, adsorption and transport to effectively address the environmental degradation of chlorinated compounds. Importantly, the particulates are synthesized through an aerosol route using sugars as precursors followed by a simple and inexpensive carbothermal reduction process without the utilization of NaBH₄. The process was first described by Mallouk, but they have not shown TCE degradation efficiency. Hoch, L. B.; Mack, E. J.; Hydutsky, B. W.; Hershman, J. M.; Skluzacek, J. M.; Mallouk, T. E., Environ. Sci. Technol. 2008, 42 (7), 2600-2605.

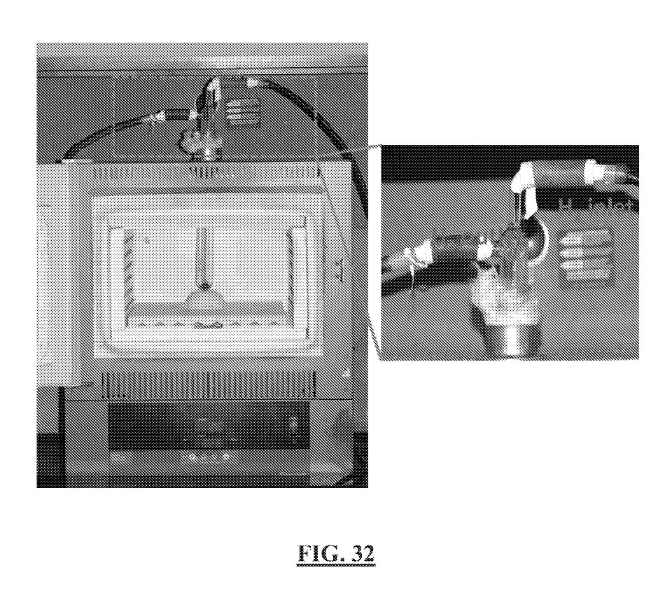

Our method of doing the carbothermal reduction follows. Here, the collected Fe₃O₄/C powder is placed in a crucible boat in a quartz tube inside a tube furnace, which was is kept at 700° C. for 10 hrs under the flowing argon. The tube is purged with Ar for 1 hr before heating and the sample is allowed to cool down to room temperature in an Ar atmosphere before removal from the tube furnace. Furthermore, a mild passivation step using deoxidized water or ethanol (95%) should be taken before the sample removal from the tube furnace. Otherwise, spontaneous ignition will happen as soon as zero-valent iron nanoparticles are in contact with air oxygen. FIG. 32 shows the schematic of the reduction apparatus. Nitrogen can be used instead of Ar.

In a variant of the technique, we pass either pure hydrogen or a hydrogen/nitrogen mixture instead of Ar. When hydrogen is used, the temperature is taken up to 500° C. and the reduction is done for 5 hours

We note again, that the carbothermal method does not always produce an active material. We do not have enough information to clearly state why this is so and under what conditions it will work. What we do know is the fact that all the materials when treated with Pd will then work well.

### 1. Carbothermal Synthesis of Aerosol-Based Multifunctional Iron-Carbon Nanocomposites

The most reactive nanoscale zero-valent iron particles are made by aqueous reduction of iron salts with sodium borohydride. However, this process involves the use of sodium borohydride and thus adds to the material costs. See Hoch, L. B.; Mack, E. J.; Hydutsky, B. W.; Hershman, J. M.; Skluzacek, J. M.; Mallouk, T. E., Environ. Sci. Technol. 2008, 42 (7), 2600-2605.

We describe here, the facile preparation of a multifunctional particulate system containing zero-valent iron, which has the requisite characteristics of reaction, adsorption and transport to effectively address the environmental degradation of chlorinated compounds. Importantly, the particulates are synthesized through an aerosol route using sugars as precursors followed by a simple and inexpensive carbothermal reduction process without the utilization of NaBH₄. In a typical synthesis, 6 g of sucrose and 5 g of FeSO₄.7H₂O were firstly dissolved in 50 mL of water. In aerosol-based technology, the resulting precursor solution was atomized to form aerosol droplets. When the aerosol droplets pass through the heating zone, preliminary solvent evaporation, dehydration/carbonization of sucrose and the formation of iron oxide occur, generating a black powder of Fe₃O₄/C composites. The temperature of the heating zone was held at 1000° C. The resulting particles were collected by filter paper maintained at 100° C. To obtain Fe/C composites, the carbothermal reduction process was employed. Here, the collected Fe₃O₄/C powder is placed in a crucible boat in a quartz tube inside a tube furnace, which was kept at 700° C. for 10 hrs under the flowing argon or nitrogen. The tube was purged with Ar or Nitrogen for 1 hr before heating began, and the sample was allowed to cool down to room temperature under the protection of Ar or Nitrogen before removing from the tube furnace. These materials are active in dechlorination.

2. Incorporating Activated Carbon and/or Carbon Black into the Materials.

Dilution of our materials with activated carbon or carbon black can significantly decrease the cost since these materials are available at low costs. Zerovalent iron can be introduced onto these well known forms of carbon or the carbons by themselves can be added to our Fe/C composites. In the case of simple dilution with just activated carbon or carbon black, reactivities will be lower but the materials will still be effective in remediating chlorinated hydrocarbons.

We can dilute our Fe/C materials with activated carbon or with carbon black. The role of the other carbons is to enhance adsorption to quickly remove TCE from solution. On a per gram of material, dilution will proportionately reduce the reaction efficiency. For example, if it takes 3 hours to remove TCE using exclusively our Fe/C materials, diluting it 9 fold (10 wt % Fe/ and 90 wt % carbon black) we believe will lead to TCE removal in 30 hours (since we are only using 1/10th the original material).

The carbon particles used by Mallouk and others (carbon black or activated carbon) are not microspheres (Schrick, B.; Hydutsky, B. W.; Blough, J. L.; Mallouk, T. E., Delivery Vehicles for Zerovalent Metal Nanoparticles in Soil and Groundwater. Chem. Mater. 2004, 16, (11), 2187-2193). They have ill-defined shapes. Ours is the only technology that makes microspheres (both the hydrothermal dehydration and the aerosol based process produce microspheres). It is our hypothesis that microspheres are more useful since they will follow flow streamlines more effectively. Additionally, it is our hypothesis that the attachment of iron is more effective with the aerosol route (not with the hydrothermal dehydration route, since this is a two step method as in all the literature). We are distinguished from prior art, such as Mallouk, in that we preferably produce exclusively microspheres and we believe our binding of iron to carbon is better.

### Porosity:

Porosity is measured through surface areas. When the aersolization is done at low temperatures, we get relatively nonporous microspheres with BET (Brunauer, Emmett, Taylor) surface areas that are small, i.e., 10 m²/g. At higher temperatures when the particles become porous, the surface areas range from 200-400 m²/g. However, we believe that they can be even higher, i.e., up to 1000 m²/g.

In the present application, since the microspheres are porous, sphericity is specified as though the microspheres are coated with a material that fills in all the pores, but does not extend beyond the pores. We measure sphericity through electron microscopy and imaging. Our sphericity measurement can be 100% due to the production by the aerosol based process and hydrothermal dehydration. However, even sphericity as low as 95%, 90%, 85%, or even 80% would allow good transport of the particles and would be an improvement over the prior art of which the inventors are aware. Our microspheres are submicron and/or micron sized particles.

By “monodisperse” we mean that substantially all particles in a sample have a diameter within 50% of all other particles, and preferably within 20% of all other particles, more preferably within 10% of all other particles, even more preferably within 5% of all other particles, and most preferably within 1% of all other particles. By “substantially all” we mean at least 80%, preferably at least 85%, more preferably at least 90%, even more preferably at least 95%, most preferably at least 99%. When lubricating, preferably substantially all of the particles are within the desired sphericity range and monodisperse.

For the TCE dechlorination application, the coating (i.e., polyelectrolyte) does extend beyond the pores and forms a layer on the surface of the particle, but the coating does not fill the pores. The pores are left open in the TCE application to allow the TCE diffuse in and adsorb to the surface of all the pores. Having micropores increases the surface area and allows more adsorption. Hence keeping the pores open is helpful.

### Spherical Carbon Particles as Lubricants:

We have demonstrated the use of spherical particles of different sizes (i.e., ranging from the nanometer to micrometer lengthscale), composition, and surface coating (e.g., surfactants, polymers, proteins) are effective oil and water-based lubricants, which lower the friction forces between shearing surfaces, even at relatively high loads. Specifically, we show that hard carbon spheres (HCS)¹, coated with a layer of sodium dodecyl sulfate (SDS) surfactant, and dispersed in water, is an effective water-based lubricant. The friction coefficient between two optically polished silica surfaces with the HCS-SDS complex acting as a lubricant is as low as 0.006 (and possibly lower). For comparison, the friction coefficient between 2 glass surfaces is 0.4², between 2 teflon surfaces is 0.04², and the friction coefficient between synovial joints is 0.01².

We see the particles being used in microelectromechanical systems (MEMS) and in microfluidics systems. The lubricants can be used in organic media, aqueous media and in ionic liquids. They can also be used as dry lubricants, for space-related applications.

These materials have tremendous use as “micro ball bearings”. They can be used in microelectromechanical devices (MEMS), in microfluidics, in space applications, etc.

The materials are very easily manufactured from a variety of sugars and polysaccharides. They can be easily coated. They can work as lubricants in dry environments, in organic liquids, in water, and in ionic liquids.

Our invention relates to the use of these materials. The materials are made by the following process:

1. Dissolve the sugars in water and heat in an autoclave (sealed vessel) for 5 hours at a temperature of 150-250 C. The sugars dehydrate partially to form spheres that are mostly carbon.

2. Pyrolize these particles in a furnace at 1000 C in an inert atmosphere (argon or nitrogen). The particles are entirely converted to carbon spheres.

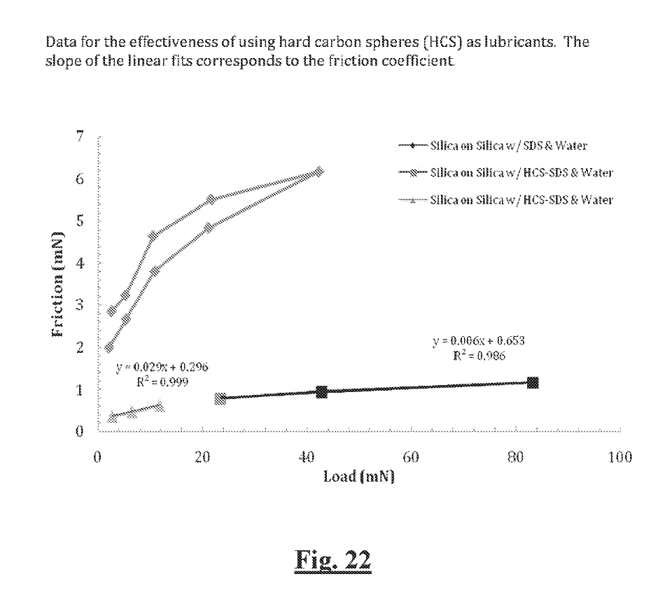

We have characterized the lubrication properties of these materials. We have samples of these materials. Experimental data available is seen in FIGS. 21 and 22, including plots of the coefficient of friction vs. load [2B, 3B]. FIG. 22 illustrates data for the effectiveness of using hard carbon spheres (HCS) as lubricants. The slope of the linear fits corresponds to the friction coefficient.

Although SDS was used in this case, several other surfactants of the chemical formula CH3(CH2)n(HG) where 3<n<21 (i.e., the length of the hydrocarbon portion, or tail group, of the surfactant can vary) and HG is the polar headgroup of the surfactant, which can be an amine, carboxylic acid, phosphonic acid, alcohol, thiol group or their respective salts with counterions such as sodium, potassium, chlorine, bromine.

The novelty in our approach is an incredibly cheap starting material (sucrose, starch, cyclodextrins, cellulose, etc.) and the uniformity and monodispersity of the particles. One would likely choose whichever starting material is the least expensive at the time. We are not able to see these precursors or the particle uniformity mentioned in prior art patents.

Another application is the possibility of using these for methane storage and to nucleate gas hydrates with these materials.

Preferably substantially all of the particles used for lubrication in a particular method are monodisperse and have good sphericity. By “monodisperse” we mean that substantially all particles in a sample have a diameter within 50% of all other particles, and preferably within 20% of all other particles, more preferably within 10% of all other particles, even more preferably within 5% of all other particles, and most preferably within 1% of all other particles. By “substantially all” we mean at least 80%, preferably at least 85%, more preferably at least 90%, even more preferably at least 95%, most preferably at least 99%. When lubricating, preferably substantially all of the particles are within the desired sphericity range and monodisperse.

We measure sphericity of the lubricating particles through electron microscopy and imaging. Our sphericity measurement can be 100% due to the production by hydrothermal dehydration. However, even sphericity as low as 95%, 90%, 85%, or even 80% would allow good lubrication and would be an improvement over the prior art of which the inventors are aware. Our lubricating microspheres are submicron or micron sized particles.

Carbon particles made by the hydrothermal dehydration and pyrolysis process are typically smooth on the surface which allows them to roll easily. When combined with the monodispersity, they become good lubricating materials. The pores if any, are extremely small—micro and nanopores. The coating (i.e., surfactant, etc.) does extend beyond the pores and actually coats the external surface. The coating provides a “cushioning” effect to the rolling and is essential for good lubrication.

### Acronyms:

Barnstead E-pure purifier IA

### Brunauer-Emmett-Teller BET

Carboxymethyl cellulose CMC

### Deionized DI

Dense nonaqueous phase liquid DNAPL
Ethyl triethoxysilane ETES
Flame ionization detector FID
Gas chromatography GC
Hard carbon spheres HCS
Microelectromechanical systems MEMS
Nanoscale zero-valent iron NZVI
Poly (acrylic acid) PAA
Reactive nanoiron particles RNIP
Scanning electron microscopy SEM
Sodium dodecyl sulfate SDS
Tetraethyl orthosilicate TEOS
Transmission electron microscopy TEM

### Trichloroethylene TCE

### Tufenkji-Elimelech T-E

Zerovalent iron ZVI

### REFERENCES

1. Dowideit, P.; von Sonntag, C., Reaction of ozone with ethene and its methyl- and chlorine-substituted derivatives in aqueous solution. Environ. Sci. Technol. 1998, 32, 1112.
2. Cowell, M.; Kibbey, T.; Zimmerman, J.; Hayes, K., Partitioning of ethoxylated nonionic surfactants in water/NAPL systems: Effects of surfactant and NAPL properties. Environ. Sci. Technol. 2000, 34, 1583.
3. Nutt, M.; Hughes, J.; Wong, M., Designing Pd-on-Au bimetallic nanoparticle catalysts for trichloroethene hydrodechlorination. Environ. Sci. Technol. 2005, 39, 1346.
4. Nyer, E. K.; Vance, D. B., Nano-scale iron for dehalogenation. Ground Water Monit. Remediat. 2001, 2, 41-46.
5. Schrick, B.; Hydutsky, B. W.; Blough, J. L.; Mallouk, T. E., Delivery Vehicles for Zerovalent Metal Nanoparticles in Soil and Groundwater. Chem. Mater. 2004, 16, (11), 2187-2193.
6. Jeen, S.; Gillham, R.; Blowes, D., Effects of carbonate precipitates on long-term performance of granular iron for reductive dechlorination of TCE. Environ. Sci. Technol. 2006, 40, 6432.
7. Wang, C. B.; Zhang, W. X., Synthesizing Nanoscale Iron Particles for Rapid and Complete Dechlorination of TCE and PCBs. Environ. Sci. Technol. 1997, 31, (7), 2154-2156.
8. Xu, Y.; Zhang, W., Subcolloidal Fe/Ag particles for reductive dehalogenation of chlorinated benzenes. Ind. Eng. Chem. Res. 2000, 39, 2238.
9. Lowry, G.; Reinhard, M., Pd-Catalyzed TCE dechlorination in groundwater: Solute effects, biological control, and oxidative catalyst regeneration. Environ. Sci. Technol. 2000, 34, 3217.
10. Schrick, B.; Blough, J. L.; Jones, A. D.; Mallouk, T. E., Hydrodechlorination of Trichloroethylene to Hydrocarbons Using Bimetallic Nickel-Iron Nanoparticles. Chem. Mater. 2002, 14, (12), 5140-5147.
11. Joo, S.; Feitz, A.; Waite, T., Oxidative degradation of the carbothioate herbicide, molinate, using nanoscale zero-valent iron. Environ. Sci. Technol. 2004, 38, 2242.
12. Tee, Y.; Grulke, E.; Bhattacharyya, D., Role of Ni/Fe nanoparticle composition on the degradation of trichloroethylene from water. Ind. Eng. Chem. Res. 2005, 44, 7062.
13. Dror, I.; Baram, D.; Berkowitz, B., Use of nanosized catalysts for transformation of chloro-organic pollutants. Environ. Sci. Technol. 2005, 39, 1283.
14. Liu, Y.; Majetich, S. A.; Tilton, R. D.; Sholl, D. S.; Lowry, G. V., TCE Dechlorination Rates, Pathways, and Efficiency of Nanoscale Iron Particles with Different Properties. Environ. Sci. Technol. 2005, 39, (5), 1338-1345.
15. Liu, Y.; Choi, H.; Dionysiou, D.; Lowry, G. V., Trichloroethene Hydrodechlorination in Water by Highly Disordered Monometallic Nanoiron. Chem. Mater. 2005, 17, (21), 5315-5322.
16. Wust, W. F.; Kober, R.; Schlicker, O.; Dahmke, A., Combined zero- and first-order kinetics model of the degradation of TCE and cis-DCE with commercial iron. Environ. Sci. Technol. 1999, 33, 4304.
17. Astrup, T.; Stipp, S.; Christensen, T., Immobilization of chromate from coal fly ash leachate using an attenuating barrier containing zero-valent iron. Environ. Sci. Technol. 2000, 34, 4163.
18. Elliott, D. W.; Zhang, W. X., Field Assessment of Nanoscale Bimetallic Particles for Groundwater Treatment. Environ. Sci. Technol. 2001, 35, (24), 4922-4926.
19. Yabusaki, S.; Cantrell, K.; Sass, B.; Steefel, C., Multicomponent reactive transport in an in situ zero-valent iron cell. Environ. Sci. Technol. 2001, 35, 1493.
20. Morrison, S., Performance evaluation of a permeable reactive barrier using reaction products as tracers. Environ. Sci. Technol. 2003, 37, 2302.
21. Casey, F.; Ong, S.; Horton, R., Degradation and transformation of trichloroethylene in miscible-displacement experiments through zerovalent metals. Environ. Sci. Technol. 2000, 34, 5023.
22. Shimotori, T.; Nuxoll, E.; Cussler, E.; Arnold, W., A polymer membrane containing Fe0 as a contaminant barrier. Environ. Sci. Technol. 2004, 38, 2264.
23. Yao, K.; Habibian, M.; O'Melia, C., Water and waste water filtration: Concepts and applications. Environ. Sci. Technol. 1971, 5, 1105.
24. Rajagopalan, R.; Tien, C., Trajectory analysis of deep-bed filtration with the sphere-in-cell porous media model. AIChE J. 1976, 22, 523.
25. Spielman, L., Particle capture from low-speed laminar flows. Annu. Rev. Fluid Mech. 1977, 9, 297.
26. Tufenkji, N.; Elimelech, M., Correlation Equation for Predicting Single-Collector Efficiency in Physicochemical Filtration in Saturated Porous Media. Environ. Sci. Technol. 2004, 38, (2), 529-536.
27. Hydutsky, B. W.; Mack, E. J.; Beckerman, B. B.; Skluzacek, J. M.; Mallouk, T. E., Optimization of Nano- and Microiron Transport through Sand Columns Using Polyelectrolyte Mixtures. Environ. Sci. Technol. 2007, 41, (18), 6418-6424.
28. He, F.; Zhao, D., Manipulating the size and dispersibility of zerovalent iron nanoparticles by use of carboxymethyl cellulose stabilizers. Environ. Sci. Technol. 2007, 41, 6216.
29. Phenrat, T.; Saleh, N.; Sirk, K.; Tilton, R. D.; Lowry, G. V., Aggregation and Sedimentation of Aqueous Nanoscale Zerovalent Iron Dispersions. Environ. Sci. Technol. 2007, 41, (1), 284-290.
30. Sayles, G. D.; You, G.; Wang, M.; Kupferle, M. J., DDT, DDD, and DDE Dechlorination by Zero-Valent Iron. Environ. Sci. Technol. 1997, 31, (12), 3448-3454.
31. Alessi, D. S.; Li, Z.; Synergistic, effect of cationic surfactants on perchloroethylene degradation by zero-valent iron. Environ. Sci. Technol. 2001, 35, 3713.
32. Quinn, J.; Geiger, C.; Clausen, C.; Brooks, K.; Coon, C.; O'Hara, S.; Krug, T.; Major, D.; Yoon, W. S.; Gavaskar, A.; Holdsworth, T., Field Demonstration of DNAPL Dehalogenation Using Emulsified Zero-Valent Iron. Environ. Sci. Technol. 2005, 39, (5), 1309-1318.
33. Saleh, N.; Phenrat, T.; Sirk, K.; Dufour, B.; Ok, J.; Sarbu, T.; Matyjaszewski, K.; Tilton, R. D.; Lowry, G. V., Adsorbed triblock copolymers deliver reactive iron nanoparticles to the oil/water interface. Nano Lett. 2005, 5, (12), 2489-94.
34. He, F.; Zhao, D., Preparation and Characterization of a New Class of Starch-Stabilized Bimetallic Nanoparticles for Degradation of Chlorinated Hydrocarbons in Water. Environ. Sci. Technol. 2005, 39, (9), 3314-3320.
35. Ditsch, A.; Laibinis, P. E.; Wang, D. I. C.; Hatton, T. A., Controlled Clustering and Enhanced Stability of Polymer-Coated Magnetic Nanoparticles. Langmuir 2005, 21, (13), 6006-6018.
36. Alessi, D. S.; Li, Z., Synergistic Effect of Cationic Surfactants on Perchloroethylene Degradation by Zero-Valent Iron. Environ. Sci. Technol. 2001, 35, (18), 3713-3717.
37. He, F.; Zhao, D.; Liu, J.; Roberts, C. B., Stabilization of Fe—Pd Nanoparticles with Sodium Carboxymethyl Cellulose for Enhanced Transport and Dechlorination of Trichloroethylene in Soil and Groundwater. Ind. Eng. Chem. Res. 2007, 46, (1), 29-34.
38. Kanel, S. R.; Goswami, R. R.; Clement, T. P.; Barnett, M. O.; Zhao, D., Two Dimensional Transport Characteristics of Surface Stabilized Zero-valent Iron Nanoparticles in Porous Media. Environ. Sci. Technol. 2008, 42, (3), 896-900.
39. Saleh, N.; Kim, H.-J.; Phenrat, T.; Matyjaszewski, K.; Tilton, R. D.; Lowry, G. V., Ionic Strength and Composition Affect the Mobility of Surface-Modified Fe0 Nanoparticles in Water-Saturated Sand Columns. Environ. Sci. Technol. 2008, 42, (9), 3349-3355.
40. Hoch, L. B.; Mack, E. J.; Hydutsky, B. W.; Hershman, J. M.; Skluzacek, J. M.; Mallouk, T. E., Carbothermal Synthesis of Carbon-supported Nanoscale Zero-valent Iron Particles for the Remediation of Hexavalent Chromium. Environ. Sci. Technol. 2008, 42, (7), 2600-2605.
41. Phenrat, T.; Saleh, N.; Sirk, K.; Kim, H.-J.; Tilton, R. D.; Lowry, G. V., Stabilization of aqueous nanoscale zerovalent iron dispersions by anionic polyelectrolytes: adsorbed anionic polyelectrolyte layer properties and their effect on aggregation and sedimentation. J. Nanopart. Res. 2008, 10, 795-814.
42. Zheng, T.; Pang, J.; Tan, G.; He, J.; McPherson, G.; Lu, Y.; John, V. T.; Zhan, J., Surfactant templating effects on the encapsulation of iron oxide nanoparticles within silica microspheres. Langmuir 2007, 23, 5143.
43. Lu, Y.; Fan, H.; Stump, A.; Ward, T. L.; Rieker, T.; Brinker, C. J., Aerosol-assisted self-assembly of mesostructured spherical nanoparticles. Nature 1999, 398.
44. Zheng, T.; Zhan, J.; Pang, J.; Tan, G.; He, J.; McPherson, G.; Lu, Y.; John, V. T., Mesoporous carbon nanocapsules from enzymatically polymerized poly(4-ethylphenol) confined in silica aerosol particles. Adv. Mater. 2006, 18, 2735.
45. Li, A.; Tai, C.; Zhao, Z.; Wang, Y.; Zhang, Q.; Jiang, G.; Hu, J., Debromination of decabrominated diphenyl ether by resin-bound iron nanoparticles. Environ. Sci. Technol. 2007, 41, 6841.
46. Zheng, T.; Zhan, J.; He, J.; Day, C.; Lu, Y.; McPherson, G. L.; Piringer, G.; John, V. T., Reactivity Characteristics of Nanoscale Zerovalent Iron&#x2212; Silica Composites for Trichloroethylene Remediation. Environ. Sci. Technol. 2008, 42, (12), 4494-4499.
47. Zheng, T.; Zhan, J.; He, J.; Day, C.; Lu, Y.; McPherson, G. L.; Piringer, G.; John, V. T., Reactivity Characteristics of Nanoscale Zerovalent Iron-Silica Composites for Trichloroethylene Remediation. Environ. Sci. Technol. 2008, 42, (12), 4494-4499.
48. Muftikian, R.; Fernando, Q.; Korte, N., A method for the rapid dechlorination of low molecular weight chlorinated hydrocarbons in water. Water Res. 1995, 29, 2434.
49. Schreier, C.; Reinhard, M., Catalytic hydrodehalogenation of chlorinated ethylenes using palladium and hydrogen for the treatment of contaminated water. Chemosphere 1995, 31, 3475.
50. Kuan-Mu Yao; Habibian, M. T.; O'Melia, C. R., Water and waste water filtration. Concepts and applications. Environ. Sci. Technol. 1971, 5, 1105-1112.
51. Zhan, J.; Zheng, T.; Piringer, G.; Day, C.; McPherson, G. L.; Lu, Y.; Papadopoulos, K.; John, V. T., Transport Characteristics of Nanoscale Functional Zerovalent Iron/Silica Composites for in Situ Remediation of Trichloroethylene. Environ. Sci. Technol. 2008, 42, (23), 8871-8876.
52. Wang, Q.; Li, H.; Chen, L. Q.; Huang, X. J., Monodispersed hard carbon spherules with uniform nanopores. Carbon 2001, 39, (14), 2211-2214.
1A. Al-abed, S. R.; Chen, J., Transport of trichloroethylene (TCE) in natural soil by electroosmosis. In Physicochemical Groundwater Remediation, Smith, J. A.; Burns, S. E., Eds. Kluwer academic/Plenum: New York, 2001; pp 91-114.
2A. Saleh, N.; Sirk, K.; Liu, Y.; Phenrat, T.; Dufour, B.; Matyjaszewski, K.; Tilton, R. D.; Lowry, G. V., Surface modifications enhance nanoiron transport and NAPL targeting in saturated porous media. Environ. Eng. Sci. 2007, 24, (1), 45-57.
3A. Mackay, D. M.; Cherry, J. A., Groundwater contamination: Pump-and treat remediation. Environ. Sci. Technol. 1989, 23, (6), 630-636.
4A. Nakano, Y.; Hua, L.; Nishijima, W.; Shoto, E.; Okada, M., Biodegradation of Trichloroethylene (TCE) Adsorbed on Granular Activated Carbon (GAC). Wat. Res. 2000, 34, (17), 4139-4142n.
5A. Ritter, K.; Odziemkowski, M. S.; Gillham, R. W., An in situ study of the role of surface films on granular iron in the permeable iron wall technology. J. Contam. Hydrol. 2002, 55, 87.
6A. Scherer, M. M.; Richter, S.; Valentine, R. L.; Alvarez, P. J. J., Chemistry and microbiology of permeable reactive barriers for in situ groundwater clean up. Crit. Rev. Environ. Sci. Technol. 2000, 30, (3), 363.
7A. O'Hannesin, S. F.; Gillham, R. W., Long-term performance of an in situ “iron wall” for remediation of VOCs. Ground Water 1998, 36, ((1)), 164.
8A. Heron, G.; Christensen, T. H.; Enfield, C. G., Henry's Law Constant for Trichloroethylene between 10 and 95° C. Environ. Sci. Technol. 1998, 32, (10), 1433-1437.
9A. Elliott, D. W.; Zhang, W. X., Field Assessment of Nanoscale Bimetallic Particles for Groundwater Treatment. Environ. Sci. Technol. 2001, 35, (24), 4922-4926.
10A. Phenrat, T.; Saleh, N.; Sirk, K.; Tilton, R.; Lowry, G., Aggregation and sedimentation of aqueous nanoscale zerovalent iron dispersions. Environ. Sci. Technol. 2007, 41, 284.
11A. Nyer, E. K.; Vance, D. B., Nano-scale iron for dehalogenation. Ground Water Monit. Remediat. 2001, 2, 41-46.
12A. Zheng, T.; Zhan, J.; He, J.; Day, C.; Lu, Y.; McPherson, G. L.; Piringer, G.; John, V. T., Reactivity characteristics of nanoscale zerovalent iron—silica composites for trichloroethylene remediation. Environ Sci Technol 2008, 42, (12), 4494-9.
13A. Quinn, J.; Geiger, C.; Clausen, C.; Brooks, K.; Coon, C.; O'Hara, S.; Krug, T.; Major, D.; Yoon, W. S.; Gavaskar, A.; Holdsworth, T., Field Demonstration of DNAPL Dehalogenation Using Emulsified Zero-Valent Iron. Environ. Sci. Technol. 2005, 39, (5), 1309-1318.
14A. Saleh, N.; Phenrat, T.; Sirk, K.; Dufour, B.; Ok, J.; Sarbu, T.; Matyjaszewski, K.; Tilton, R. D.; Lowry, G. V., Adsorbed triblock copolymers deliver reactive iron nanoparticles to the oil/water interface. Nano Lett. 2005, 5, (12), 2489-94.
15A. He, F.; Zhao, D., Preparation and Characterization of a New Class of Starch-Stabilized Bimetallic Nanoparticles for Degradation of Chlorinated Hydrocarbons in Water. Environ. Sci. Technol. 2005, 39, (9), 3314-3320.
16A. Alessi, D. S.; Li, Z., Synergistic Effect of Cationic Surfactants on Perchloroethylene Degradation by Zero-Valent Iron. Environ. Sci. Technol. 2001, 35, (18), 3713-3717.
17A. Schrick, B.; Hydutsky, B. W.; Blough, J. L.; Mallouk, T. E., Delivery vehicles for zerovalent metal nanoparticles in soil and groundwater. Chem. Mater. 2004, 16, 2187.
18A. He, F.; Zhao, D., Manipulating the size and dispersibility of zerovalent iron nanoparticles by use of carboxymethyl cellulose stabilizers. Environ. Sci. Technol. 2007, 41, 6216.
19A. Kanel, S. R.; Choi, H., The transport characteristics of polymer stabilized nano scale zero-valent iron in porous media. Water Sci. Technol. 2007, 55, 157.
20A. Lubick, N., Cap and degrade: a reactive nanomaterial barrier also serves as a cleanup tool. Environ. Sci. Technol. 2009, 43, (2), 235-235.
21A. Choi, H.; Agarwal, S.; Al-Abed, S. R., Adsorption and simultaneous dechlorination of PCBs on GAC/Fe/Pd: mechanistic aspects and reactive capping barrier concept. Environ. Sci. Technol. 2009, 43, (2), 488-93.
22A. Al-Abed, S. R.; Jegadeesan, G.; Scheckel, K. G.; Tolaymat, T., Speciation, characterization, and mobility of As, Se, and Hg in flue gas desulphurization residues. Environmental Science & Technology 2008, 42, (5), 1693-1698.
23A. Zhan, J.; Zheng, T.; Piringer, G.; Day, C.; McPherson, G. L.; Lu, Y.; Papadopoulos, K.; John, V. T., Transport Characteristics of Nanoscale Functional Zerovalent Iron/Silica Composites for in Situ Remediation of Trichloroethylene. Environ. Sci. Technol. 2008, 42, (23), 8871-8876.
24A. Schreier, C.; Reinhard, M., Catalytic hydrodehalogenation of chlorinated ethylenes using palladium and hydrogen for the treatment of contaminated water. Chemosphere 1995, 31, 3475.
25A. Wang, C.; Zhang, W., Synthesizing nanoscale iron particles for rapid and complete dechlorination of TCE and PCBs. Environ. Sci. Technol. 1997, 31, (7), 2154.
26A. Muftikian, R.; Fernando, Q.; Korte, N., A method for the rapid dechlorination of low molecular weight chlorinated hydrocarbons in water. Water Res. 1995, 29, 2434.
27A. Tufenkji, N.; Elimelech, M., Correlation equation for predicting single-collector efficiency in physicochemical filtration in saturated porous media. Environ. Sci. Technol. 2004, 38, (2), 529.
28A. He, F.; Zhao, D.; Liu, J.; Roberts, C. B., Stabilization of Fe—Pd Nanoparticles with Sodium Carboxymethyl Cellulose for Enhanced Transport and Dechlorination of Trichloroethylene in Soil and Groundwater. Ind. Eng. Chem. Res. 2007, 46, (1), 29-34.
29A. Wang, Q.; Li, H.; Chen, L.; Huang, X., Monodispersed hard carbon spherules with uniform nanopores. Carbon 2001, 39, (14), 2211-2214.
30A. Yang, R.; Qiu, X.; Zhang, H.; Li, J.; Zhu, W.; Wang, Z.; Huang, X.; Chen, L., Monodispersed hard carbon spherules as a catalyst support for the electrooxidation of methanol. Carbon 2005, 43, (1), 11-16.
31A. Wang, Q.; Li, H.; Chen, L. Q.; Huang, X. J., Novel spherical microporous carbon as anode material for Li-ion batteries. Solid State Ionics 2002, 152, 43-50.
32A. Liu, S. P.; Chen, S.; Liu, Z. F.; Hu, X. L.; Li, T. S., Resonance Rayleigh scattering spectra of interaction of sodium carboxymethylcellulose with cationic acridine dyes and their analytical applications. Analytica Chimica Acta 2005, 535, (1-2), 169-175.
33A. Simon, S.; Dugast, J. Y.; Le Cerf, D.; Picton, L.; Muller, G., Amphiphilic polysaccharides. Evidence for a competition between intra and intermolecular associations in dilute system. Polymer 2003, 44, (26), 7917-7924.
34A. Bacchin, P.; Bonino, J. P.; Martin, F.; Combacau, M.; Barthes, P.; Petit, S.; Ferret, J., Surface pre-coating of talc particles by carboxylmethyl cellulose adsorption: Study of adsorption and consequences on surface properties and settling rate. Colloids and Surfaces A: Physicochem. Eng. Aspects 2006, 272, (3), 211-219.
35A. Pawlik, M., Polymeric dispersants for coal-water slurries. Colloids and Surfaces a-Physicochemical and Engineering Aspects 2005, 266, (1-3), 82-90.
36A. He, F.; Zhao, D., Hydrodechlorination of trichloroethene using stabilized Fe—Pd nanoparticles: Reaction mechanism and effects of stabilizers, catalysts, and reaction conditions. Appl. Catal. B 2008, 84, 533.
37A. Hoch, L. B.; Mack, E. J.; Hydutsky, B. W.; Hershman, J. M.; Skluzacek, I. M.; Mallouk, T. E., Carbothermal synthesis of carbon-supported nanoscale zero-valent iron particles for the remediation of hexavalent chromium. Environmental Science & Technology 2008, 42, (7), 2600-2605.
38A. Liao, C. H.; Kang, S. F.; Hsu, Y. W., Zero-valent iron reduction of nitrate in the presence of ultraviolet light, organic matter and hydrogen peroxide. Water Research 2003, 37, (17), 4109-4118.
39A. Lien, H. L.; Zhang, W. X., Nanoscale Pd/Fe bimetallic particles: Catalytic effects of palladium on hydrodechlorination. Applied Catalysis B-Environmental 2007, 77, (1-2), 110-116.
40A. Phenrat, T.; Liu, Y. Q.; Tilton, R. D.; Lowry, G. V., Adsorbed Polyelectrolyte Coatings Decrease Fe-0 Nanoparticle Reactivity with TCE in Water: Conceptual Model and Mechanisms. Environmental Science & Technology 2009, 43, (5), 1507-1514.
41A. Phenrat, T.; Saleh, N.; Sirk, K.; Tilton, R. D.; Lowry, G. V., Aggregation and Sedimentation of Aqueous Nanoscale Zerovalent Iron Dispersions. Environ. Sci. Technol. 2007, 41, (1), 284-290.
42A. Hoch, L. B.; Mack, E. J.; Hydutsky, B. W.; Hershman, J. M.; Skluzacek, J. M.; Mallouk, T. E., Carbothermal Synthesis of Carbon-supported Nanoscale Zero-valent Iron Particles for the Remediation of Hexavalent Chromium. Environ. Sci. Technol. 2008, 42, (7), 2600-2605.
43A. ASTM Standard D 4187-82, Zeta potential of colloids in water and waste water. In American Society for Testing and Materials, West Conshohocken, Pa., 1985.
44A. Kuan-Mu Yao; Habibian, M. T.; O'Melia, C. R., Water and waste water filtration. Concepts and applications. Environ. Sci. Technol. 1971, 5, 1105-1112.
45A. Tufenkji, N.; Elimelech, M., Correlation Equation for Predicting Single-Collector Efficiency in Physicochemical Filtration in Saturated Porous Media. Environ. Sci. Technol. 2004, 38, (2), 529-536.
46A. Saleh, N.; Kim, H.-J.; Phenrat, T.; Matyjaszewski, K.; Tilton, R. D.; Lowry, G. V., Ionic Strength and Composition Affect the Mobility of Surface-Modified Fe0 Nanoparticles in Water-Saturated Sand Columns. Environ. Sci. Technol. 2008, 42, (9), 3349-3355.
47A. Phenrat, T.; Kim, H.-J.; Fagerlund, F.; Illangasekare, T.; Tilton, R. D.; Lowry, G. V., Particle Size Distribution, Concentration, and Magnetic Attraction Affect Transport of Polymer-Modified Fe0 Nanoparticles in Sand Columns. Environmental Science & Technology ASAP.
48A. Cantrell, K. J.; Kaplan, D. I.; Gilmore, T. J., Injection of colloidal Fe-0 particles in sand with shear-thinning fluids. Journal of Environmental Engineering-Asce 1997, 123, (8), 786-791.
1B. Wang, Q.; Li, H.; Chen, L. Q.; Huang, X. J. Carbon 2001, 39, 2211-2214.
2B. Http://encarta.msn.com/media_{—}461544499/Table_of_Friction_Coefficients.html
3B. Coefficients of Friction. Retrieved Oct. 12, 2009, from Physlink Website: http://www.physlink.com/reference/FrictionCoefficients.cfm.
1C. Mansoori, G. A.; Bastami, T. R.; Ahmadpour, A.; Eshaghi, Z., Environmental application of nanotechnology. In Annual Review of Nano Research, Cao, G.; Brinker, C. J., Eds. World Scientific Publishing Company: 2008; Vol. 2, pp 439-493.
2C. Tratnyek, P. G.; Johnson, R. L., Nanotechnologies for environmental cleanup. Nano Today 2006, 1, (2), 44-48.
3C. Mauter, M. S.; Elimelech, M., Environmental applications of carbon-based nanomaterials. Environ. Sci. Technol. 2008, 42, (16), 5843-5859.
4C. Yoon, K.; Hsiao, B. S.; Chu, B., Functional nanofibers for environmental applications. J. Mater. Chem. 2008, 18, (44), 5326-5334.
5C. Zhang, H.; Chen, G.; Bahnemann, D. W., Photoelectrocatalytic materials for environmental applications. J. Mater. Chem. 2009, 19, (29), 5089-5121.
6C. Arnold, W. A.; Roberts, A. L., Pathways of chlorinated ethylene and chlorinated acetylene reaction with Zn(0). Environ. Sci. Technol. 1998, 32, (19), 3017-3025.
7C. Chen, S.-S.; Hsu, H.-D.; Li, C.-W., A new method to produce nanoscale iron for nitrate removal. J. Nanopart. Res 2004, 6, (6), 639-647.
8C. Li, X. G.; Takahashi, S.; Watanabe, K.; Kikuchi, Y.; Koishi, M., Hybridization and characteristics of Fe and Fe—Co nanoparticles with polymer particles. Nano Lett. 2001, 1, (9), 475-480.
9C. Hoch, L. B.; Mack, E. J.; Hydutsky, B. W.; Hershman, J. M.; Skluzacek, J. M.; Mallouk, T. E., Carbothermal synthesis of carbon-supported nanoscale zero-valent iron particles for the remediation of hexavalent chromium. Environ. Sci. Technol. 2008, 42, (7), 2600-2605.
10C. Wang, C. B.; Zhang, W. X., Synthesizing Nanoscale Iron Particles for Rapid and Complete Dechlorination of TCE and PCBs. Environ. Sci. Technol. 1997, 31, (7), 2154-2156.
11C. Nyer, E. K.; Vance, D. B., Nano-scale iron for dehalogenation. Ground Water Monit. Remediat. 2001, 2, 41-46.
12C. Zhang, W. X., Nanoscale iron particles for environmental remediation: an overview. J. Nanopart. Res. 2003, 5, 323.
13C. Sun, Y. P.; Li, X. Q.; Cao, J.; Zhang, W. X.; Wang, H. P., Characterization of zero-valent iron nanoparticles. Adv. Colloid Interface Sci. 2006, 120, 47.
14C. Sarathy, V.; Tratnyek, P. G.; Nurmi, J. T.; Baer, D. R.; Amonette, J. E.; Chun, C. L.; Penn, R. L.; Reardon, E. J., Aging of iron nanoparticles in aqueous solution: effects on structure and reactivity. J. Phys. Chem. C 2008, 112, (7), 2286-2293.
15C. Liu, Y.; Choi, H.; Dionysiou, D.; Lowry, G. V., Trichloroethene hydrodechlorination in water by highly disordered monometallic nanoiron. Chem. Mater. 2005, 17, (21), 5315-5322.
16C. Schrick, B.; Blough, J. L.; Jones, A. D.; Mallouk, T. E., Hydrodechlorination of trichloroethylene to hydrocarbons using bimetallic nickel-iron nanoparticles. Chem. Mater. 2002, 14, (12), 5140-5147.
17C. Phenrat, T.; Saleh, N.; Sirk, K.; Tilton, R.; Lowry, G., Aggregation and sedimentation of aqueous nanoscale zerovalent iron dispersions. Environ. Sci. Technol. 2007, 41, 284.
18C. He, F.; Zhao, D., Manipulating the size and dispersibility of zerovalent iron nanoparticles by use of carboxymethyl cellulose stabilizers. Environ. Sci. Technol. 2007, 41, 6216.
19C. Mackenzie, K.; Schierz, A.; Georgi, A.; Kopinke, F.-D., Colloidal activated carbon and carbo-iron novel materials for In-Situ groundwater treatment. Global NEST Journal 2008, 10, (1), 54-61.
20C. Yao, K. M.; Habibian, M. M.; Omelia, C. R., Water and waste water filtration: Concepts and applications. Environ. Sci. Technol. 1971, 5, 1105.
21C. Tufenkji, N.; Elimelech, M., Correlation equation for predicting single-collector efficiency in physicochemical filtration in saturated porous media. Environ. Sci. Technol. 2004, 38, (2), 529.
22C. Schrick, B.; Hydutsky, B. W.; Blough, J. L.; Mallouk, T. E., Delivery vehicles for zerovalent metal nanoparticles in soil and groundwater. Chem. Mater. 2004, 16, 2187.
23C. Zhan, J.; Zheng, T.; Piringer, G.; Day, C.; McPherson, G. L.; Lu, Y.; Papadopoulos, K.; John, V. T., Transport characteristics of nanoscale functional zerovalent iron/silica composites for in Situ remediation of trichloroethylene. Environ. Sci. Technol. 2008, 42, (23), 8871-8876.
24C. Saleh, N.; Phenrat, T.; Sirk, K.; Dufour, B.; Ok, J.; Sarbu, T.; Matyjaszewski, K.; Tilton, R. D.; Lowry, G. V., Adsorbed triblock copolymers deliver reactive iron nanoparticles to the oil/water interface. Nano Lett. 2005, 5, (12), 2489-94.
25C. Alessi, D. S.; Li, Z., Synergistic Effect of Cationic Surfactants on Perchloroethylene Degradation by Zero-Valent Iron. Environ. Sci. Technol. 2001, 35, (18), 3713-3717.
26C. Kanel, S. R.; Choi, H., The transport characteristics of polymer stabilized nano scale zero-valent iron in porous media. Water Sci. Technol. 2007, 55, 157.
27C. Quinn, J.; Geiger, C.; Clausen, C.; Brooks, K.; Coon, C.; O'Hara, S.; Krug, T.; Major, D.; Yoon, W. S.; Gavaskar, A.; Holdsworth, T., Field demonstration of DNAPL dehalogenation using emulsified zero-valent iron. Environ. Sci. Technol. 2005, 39, (5), 1309-1318.
28C. He, F.; Zhao, D., Preparation and characterization of a new class of starch-stabilized bimetallic nanoparticles for degradation of chlorinated hydrocarbons in water. Environ. Sci. Technol. 2005, 39, (9), 3314-3320.
29C. Phenrat, T.; Liu, Y. Q.; Tilton, R. D.; Lowry, G. V., Adsorbed polyelectrolyte coatings decrease Fe-0 nanoparticle reactivity with TCE in water: conceptual model and mechanisms. Environ. Sci. Technol. 2009, 43, (5), 1507-1514.
30C. Phenrat, T.; Long, T. C.; Lowry, G. V.; Veronesi, B.; Partial, aging”) and surface modification decrease the toxicity of nanosized zerovalent iron. Environ. Sci. Technol. 2009, 43, 195.
31C. Choi, H.; Al-Abed, S. R.; Agarwal, S.; Dionysiou, D. D., Synthesis of reactive nano-Fe/Pd bimetallic system-impregnated activated carbon for the simultaneous adsorption and dechlorination of PCBs. Chem. Mater. 2008, 20, (11), 3649-3655.
32C. Lubick, N., Cap and degrade: a reactive nanomaterial barrier also serves as a cleanup tool. Environ. Sci. Technol. 2009, 43, (2), 235-235.
33C. Choi, H.; Agarwal, S.; Al-Abed, S. R., Adsorption and simultaneous dechlorination of PCBs on GAC/Fe/Pd: mechanistic aspects and reactive capping barrier concept. Environ. Sci. Technol 2009, 43, (2), 488-493.
34C. Zhan, J.; Sunkara, B.; Le, L.; John, V. T.; He, J.; McPherson, G. L.; Piringer, G.; Lu, Y., Multifunctional Colloidal Particles for in Situ Remediation of Chlorinated Hydrocarbons. Environmental Science & Technology 2009, 43, (22), 8616-8621.
35C. Lu, Y.; Fan, H.; Stump, A.; Ward, T.; Rieker, T.; Brinker, C., Aerosol-assisted self-assembly of mesostructured spherical nanoparticles. Nature 1999, 398, 223.
36C. Zheng, T.; Pang, J.; Tan, G.; He, J.; McPherson, G.; Lu, Y.; John, V. T.; Zhan, J., Surfactant templating effects on the encapsulation of iron oxide nanoparticles within silica microspheres. Langmuir 2007, 23, 5143.
37C. Zheng, T.; Zhan, J.; He, J.; Day, C.; Lu, Y.; McPherson, G. L.; Piringer, G.; John, V. T., Reactivity characteristics of nanoscale zerovalent iron/silica composites for trichloroethylene remediation. Environ. Sci. Technol. 2008, 42, (12), 4494-4499.
38C. Zhan, J.; Sunkara, B.; Le, L.; John, V. T.; He, J.; McPherson, G. L.; Piringer, G.; Lu, Y., Multifunctional colloidal particles for in situ remediation of chlorinated hydrocarbons. Environ. Sci. Technol. 2009, 43, (22), 8616-8621.
39C. Sunkara, B.; Zhan, J.; He, J.; McPherson, G. L.; Piringer, G.; John, V. T., Nanoscale zerovalent iron supported on uniform carbon microspheres for the in situ remediation of chlorinated hydrocarbons. ACS Appl. Mater. Interfaces 2010, ASAP.
40C. Nurmi, J. T.; Tratnyek, P. G.; Sarathy, V.; Baer, D. R.; Amonette, J. E.; Pecher, K.; Wang, C. M.; Linehan, J. C.; Matson, D. W.; Penn, R. L.; Driessen, M. D., Characterization and properties of metallic iron nanoparticles: Spectroscopy, electrochemistry, and kinetics. Environ. Sci. Technol. 2005, 39, 1221.
41C. Lien, H. L.; Zhang, W. X., Nanoscale Pd/Fe bimetallic particles: Catalytic effects of palladium on hydrodechlorination. Appl. Catal. B-Environ. 2007, 77, (1-2), 110-116.
42C. Phenrat, T.; Liu, Y.; Tilton, R. D.; Lowry, G. V., Adsorbed polyelectrolyte coatings decrease Fe(0) nanoparticle reactivity with TCE in water: conceptual model and mechanisms. Environ. Sci. Technol. 2009, 43, (5), 1507-14.
43C. Cantrell, K. J.; Kaplan, D. I., Injection of colloidal Fe⁰ particles in sand with shear-thinning fluids. J. Environ. Eng. 1997, 123, (8), 786.
44C. He, F.; Zhao, D., Hydrodechlorination of trichloroethene using stabilized Fe—Pd nanoparticles: Reaction mechanism and effects of stabilizers, catalysts, and reaction conditions. Appl. Catal. B 2008, 84, 533.
45C. He, F.; Zhao, D.; Liu, J.; Roberts, C. B., Stabilization of Fe—Pd Nanoparticles with Sodium Carboxymethyl Cellulose for Enhanced Transport and Dechlorination of Trichloroethylene in Soil and Groundwater. Ind. Eng. Chem. Res. 2007, 46, (1), 29-34.

All measurements disclosed herein are at standard temperature and pressure, at sea level on Earth, unless indicated otherwise. All materials used or intended to be used in a human being are biocompatible, unless indicated otherwise.

The foregoing embodiments are presented by way of example only; the scope of the present invention is to be limited only by the following claims.

1.-24. (canceled)

25. A method of remediating chlorinated hydrocarbons which are dense non aqueous phase liquids, comprising:
a) attaching zerovalent iron nanoparticles to carbon microspheres; and
b) contacting the carbon supported zerovalent iron nanoparticles with a substance containing the chlorinated hydrocarbons.

26.-76. (canceled)

77. A decontamination composition for remediation of chlorinated hydrocarbons which are dense nonaqueous phase liquids, comprising:
a) carbon microspheres;
b) zerovalent iron nanoparticles attached to the carbon microspheres.

78.-102. (canceled)

103. The composition of claim 77, further comprising a polyelectrolyte in which the carbon is enveloped and wherein the zerovalent iron nanoparticles are attached to the polyelectrolyte.

104.-116. (canceled)

117. The composition of claim 103, wherein the polyelectrolyte is from the group consisting of carboxymethyl cellulose, Starch, Dextran, poly lactate, poly ascorbate, modified chitosan, gelatin, xantham gum, poly(acrylic acid) and poly(styrene sulfonate).

118.-126. (canceled)

127. A method of remediating chlorinated hydrocarbons which are dense non-aqueous phase liquids, comprising:
a) providing nanoscale zerovalent iron/carbon particles stabilized with hydrophilic or amphiphilic organic species; and
b) combining the stabilized nanoscale zerovalent iron/carbon particles with the chlorinated hydrocarbons.

128.-134. (canceled)

135. The method of claim 127, wherein the nanoscale zerovalent iron/carbon particles adsorb and break down the chlorinated hydrocarbons.

136.-153. (canceled)

154. The method of claim 127, wherein step “b” is carried out by injecting the particles into groundwater so that the particles migrate by groundwater flow through soil and porous media and reach the sites of contamination by chlorinated hydrocarbons, where the particles partition the chlorinated hydrocarbons phase and sequester and break down the chlorinated hydrocarbons.

155.-186. (canceled)

187. A method of preparing carbon supported zerovalent iron particles for environmental remediation of chlorinated hydrocarbons which are dense non-aqueous phase liquids through use of an aerosol reactor or a spray drier comprising the steps of:
a) providing a feed stream including a carbon source;
b) adding an iron precursor to the feed stream;
c) passing the feed stream through a nozzle for aerosolization or spray.

188. The method of claim 187, further comprising the steps of:
d) creating particles by passing the feed stream through a heated zone for dehydration;
e) collecting the particles on a filter;
f) dispersing the particles in an aqueous solution;
g) adding a reducing agent to the aqueous solution; and
h) adding a polyelectrolyte to the aqueous solution, wherein the feed stream includes a monosaccharide or polysaccharide and a dilute acid.

189. The method of claim 188, wherein the reducing agent is from the group consisting of sodium borohydride, hydrazine, and a polyphenol.

190. The method of claim 188, wherein the monosaccharide or polysaccharide is from the group consisting of sucrose, glucose, cellulose, and cyclodextrins.

191. The method of claim 188, wherein the polyelectrolyte is from the group consisting of carboxymethyl cellulose, starch, dextran, poly lactate, poly ascorbate, modified chitosan, gelatin, xantham gum, poly(acrylic acid) and poly (styrene sulfonate).

192. The method of claim 188, wherein the dilute acid is sulfuric acid or nitric acid.

193. The method of claim 187, wherein the chlorinated hydrocarbon is from the group consisting of trichloroethylene, tetrachloroethene, 1,1-dichloroethene, cis- and trans-1,2-dichloroethene, and vinyl chloride.

194.-198. (canceled)

199. Carbon supported zerovalent iron particles produced by the method of claim 187.

200.-235. (canceled)

236. The method of claim 188, further comprising a catalyst.

237. The method of claim 236, wherein the catalyst is a transition metal.

238. The method of claim 236, wherein the catalyst is from the group consisting of palladium, platinum, gold, and nickel.

239. The method of claim 127, wherein the hydrophilic or amphiphilic organic species are from the group consisting of surfactants, vegetable oils, starch, and polyelectrolytes.

240. The method of claim 127, wherein the hydrophilic or amphiphilic organic species are polyelectrolytes from the group consisting of carboxymethyl cellulose (CMC) and poly (acrylic acid) (PAA), or triblock copolymers.

241.-247. (canceled)
